(19) United States
(12) Patent Application Publication
(10) Pub. No.: US 2010/0166669 A1
(43) Pub. Date: Jul. 1, 2010

(54) METHODS OF IMAGING INFLAMMATION IN PANCREATIC ISLETS

(75) Inventors: Diane J. Mathis, Brookline, MA (US); Ralph Weissleder, Peabody, MA (US); Umar Mahmood, Winchester, MA (US); Christophe O. Benoist, Brookline, MA (US)

Correspondence Address: FISH & RICHARDSON PC, P.O. BOX 1022, MINNEAPOLIS, MN, 55440-1022, US

(73) Assignees: JOSLIN DIABETES CENTER, INC., Boston, MA (US); THE GENERAL HOSPITAL CORPORATION, Boston, MA (US)

(21) Appl. No.: 11/993,373

(22) PCT Filed: Jun. 28, 2006
(86) PCT No.: PCT/US2006/025108

§ 371 Date: Mar. 11, 2010

Related U.S. Application Data
(60) Provisional application No. 60/694,602, filed on Jun. 28, 2005.

(51) Int. Cl. A61K 49/00

(52) U.S. Cl. 424/9.322; 424/9.3

(57) ABSTRACT

Described are non-invasive methods for imaging pancreatic inflammation in living mammals using Magnetic Nanoparticle Probes (MNPs).

### CLAIM OF PRIORITY

This application claims the benefit under 35 USC §119(e) of U.S. Provisional Patent Application Ser. No. 60/694,602, filed on Jun. 28, 2005, the entire contents of which are hereby incorporated by reference.

### FEDERALLY SPONSORED RESEARCH OR DEVELOPMENT

This invention was made with Government support under Grant Nos. 5RO1DK059658, 2P30DK36836 and PO1AI54904, and P50CA86355 and R24CA92782 awarded by the National Institutes of Health. The Government has certain rights in the invention.

### TECHNICAL FIELD

This invention relates to methods of imaging inflammation in pancreatic islets in type-1 diabetes mellitus.

### BACKGROUND

Type-1 diabetes (T1D) is an autoimmune disorder, generally thought to be the result of T lymphocyte attack on the insulin-producing β cells of the islets of Langerhans of the pancreas. Disease unfolds through the following two main stages: an occult phase, termed insulitis, when a mixed population of leukocytes invades the islets, promoting β-cell death; and the overt phase, diabetes, when the bulk of β cells has been destroyed and the pancreas can no longer produce sufficient insulin to control blood-glucose levels.

A major hindrance to the study of T1D is that its overt manifestation indicates that most of the islet β cells have already been destroyed and that most of the preceding, autoimmune, processes have already played out. Experimentally, in animal models, this engenders difficulties in charting the early events and unraveling their underlying mechanisms, especially given that individual animals exhibit significant scatter in disease parameters and variability in disease course. Clinically, diagnosis at such a late stage creates problems for providing effective therapy because, by that time, options for disease reversal are already severely limited, and because analogous disease heterogeneity precludes earlier “blind” intervention.

### SUMMARY

The present invention is based, at least in part, on the discovery that Magnetic Nanoparticle Probes (MNPs), including Monocrystalline Iron Oxide Nanoparticles (MIONs) and derivatives thereof (e.g., particles that have Cross-Linked and aminated dextran coating around the Iron Oxide core, or CLIOs), can be used to non-invasively image inflammation in the pancreas of a living mammal.

Thus, described herein are new methods that can be used to non-invasively monitor the initiation, progression, and reversal of insulitis in living mammals, in vivo in real time. This methodology allows, on an individual basis, the direct correlation of immunological and metabolic parameters (such as serum autoantibody levels, blood glucose levels, insulin levels, C-peptide levels, etc.) with the evolution of the insulitic lesion, either during the natural unfolding of disease or subsequent to therapeutic intervention. The methods can also be used to predict whether and when overt diabetes will develop in the future. Given the published success and non-toxicity of MNPs in the human context (see, e.g., Harisinghani et al., N. Engl. J. Med., 348:2491-2499 (2003)), application of this methodology to type-1 diabetes in human subjects is highly desirable. In general, the methods described herein are non-invasive, i.e., are performed in an intact living mammal.

In one aspect, the invention includes methods for imaging inflammation in a pancreatic tissue of a living mammal in vivo. The methods include administering a detectable amount of a composition comprising Magnetic Nanoparticle Probes (MNPs) or derivatives thereof to a living mammal; and detecting MNPs in the pancreatic tissue of the mammal, thereby imaging inflammation in the pancreatic tissue. The presence of MNPs in the pancreatic tissue of the mammal is an indication of the presence of inflammation in the tissue.

In some embodiments, the MNPs have no targeting moiety.

In some embodiments, the MNPs are detected by NMR imaging.

In some embodiments, the living mammal is at risk of developing type-1 diabetes, e.g., is selected on the basis of being at risk of developing type-1 diabetes. In some embodiments, the amount of MNPs detected in the pancreatic tissue of the mammal indicates that the mammal is developing or will develop type-1 diabetes.

In some embodiments, the living mammal is at risk of developing insulitis, e.g., is selected on the basis of being at risk of developing insulitis. In some embodiments, the amount of MNPs detected in the pancreatic tissue of the mammal indicates that the mammal is developing or will develop insulitis.

Further, the invention includes methods for evaluating the efficacy of a candidate treatment for pancreatic inflammation in a living mammal. The methods include selecting a subject; administering a candidate therapeutic intervention to the subject, e.g., a candidate therapeutic compound; and obtaining an in vivo image of inflammation in a pancreatic tissue of the subject, e.g., by a method described herein, e.g., after, e.g., less than a month after, administration of the therapeutic intervention. The presence, absence, or level of inflammation in the pancreatic tissue is indicative of the efficacy of the candidate treatment, e.g., in some embodiments, the absence or level (e.g., a decreased level) of inflammation indicates that the candidate treatment is an effective treatment for pancreatic inflammation.

In some embodiments, the methods further include obtaining an in vivo image of inflammation in a pancreatic tissue in the mammal before administration of the candidate treatment, e.g., to establish a baseline.

In some embodiments, the treatment is a treatment for type-1 diabetes, e.g., a treatment for type-1 diabetes that prevents or delays the onset or progression of type-1 diabetes.

The invention also includes methods that can be used to evaluate the efficacy of a candidate immunotherapy treatment for pancreatic cancer. The methods include selecting a subject; administering a candidate immunotherapeutic intervention to the subject, e.g., a subject who has pancreatic cancer or a control subject; and obtaining an in vivo image of inflammation in a pancreatic tissue of the subject, e.g., by a method described herein, e.g., after, e.g., less than a month after, administration of the therapeutic intervention. The presence, absence, or level of inflammation in the pancreatic tissue is indicative of the efficacy of the candidate treatment, e.g., in some embodiments, the presence or level (e.g., an increased level) of inflammation indicates that the candidate immunotherapy is an effective treatment for pancreatic cancer, as increased inflammation could indicate that the body is mounting an immune response to the cancer.

In some embodiments, the subject is at risk of developing type-1 diabetes, e.g., is selected on the basis of being at risk of developing type-1 diabetes. In some embodiments, the amount of MNPs detected in the pancreatic tissue of the subject indicates that the subject is developing or will develop type-1 diabetes.

In some embodiments, the subject is at risk of developing insulitis, e.g., is selected on the basis of being at risk of developing insulitis. In some embodiments, the amount of MNPs detected in the pancreatic tissue of the subject indicates that the subject is developing or will develop insulitis.

In another aspect, the invention provides methods for evaluating pancreatic inflammation in a subject. The methods include administering a detectable amount of a composition comprising Magnetic Nanoparticle Probes (MNPs) or derivatives thereof to the mammal; obtaining a magnetic resonance image (MRI) of MNPs in a pancreatic tissue of the mammal; and deriving a value relevant to inflammation from the image of MNPs; thereby evaluating pancreatic inflammation in the subject.

In some embodiments, the MRI is obtained immediately after administration of the MNPs.

In some embodiments, the methods further include obtaining a second in vivo image, e.g., at least about 24 hours after administration of the MNPs.

In some embodiments, the value relevant to inflammation is selected from the group consisting of vascular volume fraction (VVF), vascular leak, T1, T2, and T2*, and magnetic susceptibility.

In some embodiments, the methods include comparing the relevant value with a reference value, e.g., a reference value is derived from an image of MNPs in the pancreatic tissue of the mammal obtained previously, or a reference that represents a particular physiological state, e.g., insulitis, diabetes, or normal. In some embodiments, the relevant value as compared with the reference value is indicative of a rate of progression of pancreatic inflammation in the subject. The methods can include obtaining a plurality of in vivo images to follow the progression of insulitis in a subject over time.

In some embodiments, the MNPs are monocrystalline superparamagnetic iron oxide particles with a dextran coating, e.g., Monocrystalline Iron Oxide Nanoparticles (MIONs), or derivatives thereof.

As used herein, a “subject” is a living mammal, e.g., a human or non-human mammal. In some embodiments, the subject is a human or veterinary patient, or a subject in a clinical trial. In some embodiments, the subject is an experimental animal.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a set of three fluorescent photomicrographs of CLIO probe accumulation, reflective of microvascular leakage, as an accompaniment to islet infiltration. Islets were identified by DTZ staining (red in original, indicated by arrow in middle panel and medium grey areas in right panel), and signal from the CLIO probe (green in original, light grey areas in left and middle panels) was visualized and quantitated.

FIG. 1B is a pair of panels showing a fluorescent photomicrograph (top panel) and regions of interest (ROI) that represent islets (identified by DTZ staining) or exocrine tissue (an area selected as distinct from islets and major vessels), bottom panel.

FIG. 1C is a graph illustrating the results of a quantitative comparison of insulitic and non-insulitic pancreata. Four, 4-week-old BDC2.5/NOD (open circles) and thirteen age-matched NOD-RAG^{0/0} animals (filled triangles) were used. Symbols represent the MFI of individual animals calculated as the average MFI of all the islets (4-10) acquired per animal. The bar represents the average value.

FIG. 2A is a pair of photomicrographs of a non-infiltrated versus an infiltrated islet. Images are representative of those derived from at least two animals of each genotype. CLIO=light grey (green in original); Hst=medium grey (blue in original). The mass of medium grey (blue in original) nuclei corresponds to lymphocytic infiltrate, as identified histologically. 25× objective.

FIG. 2B is a pair of photomicrographs taken at higher magnifications. Images reveal the uptake of CLIO-Alexa-488 (light grey; green in original) by cells of macrophage-like morphology. Nuclei of infiltrating, mostly lymphoid, cells appear medium grey (blue in original) with Hst. 40× objective.

FIG. 2C is a pair of histograms and a pair of scatter graphs showing the results of identification of the cellular repository by flow cytometry. CLIO-positive cells were gated as indicated in the upper panels and staining by anti-CD11b and CD11c reagents was displayed. A group of three 4-week-old female BDC2.5/NOD mice was used per sample. One group was left uninjected (No CMFN, left column) while the other was iv injected with 10 mg/kg CLIO-Alexa-488 (+CMFN, right column).

FIG. 3A is a set of six photomicrographs illustrating the initiation of insulitis in pancreata of 2- or 3-week-old female BDC2.5/NOD (left column) or NOD-RAG^{0/0} (middle column) mice injected with CLIO-Alexa-488 (left and middle columns) or PBS (right column) and DTZ, processed and imaged ex vivo. Images were taken with a 10× objective.

FIG. 3B is a trio of graphs illustrating the evolution of insulitis. Animals ranged in age from 2-15 weeks, as indicated in the legend. Symbols represent values for individual islets (3Bi) or averages of all islet values for individual animals (3Bii). Average MFIs of CLIO signal for pancreata of individual animals were correlated with the fraction of the animal's islets that were infiltrated, assessed from (H&E)-stained sections of the same pancreas used for imaging (3Biii).

FIG. 3Ci-v is a set of five photomicrographs illustrating the composition and appearance of the infiltrate. Pancreata from CLIO/DTZ-injected NOD variants were protected from insulitis due to a lack of α:β T and B cells (3Ci) or to an augmentation of regulatory T cell numbers or activity (3Cii); or from CLIO/DTZ-injected BDC2.5 TCR transgenic mice on different genetic backgrounds promoting greater insulitis aggressivity in the order BDC2.5/NOD (3Ciii)<BDC2.5/B6.H-2g^{g7g7} (3Civ)<BDC2.5/NOD-RAG^{0/0} (3Cv). Images are with 10× objective.

FIG. 3D is a pair of graphs illustrating the results of image quantitation. MFIs surrounding the islets were plotted (3Di) and correlated with insulitis intensity revealed by H&E histological analysis (3Dii).

FIGS. 4A and 4B are graphs of MFIs of CMFN signal associated with pancreatic islets of insulitic NOD (diamonds) vs. non-insulitic NOD-RAG0/0 (circles) female mice. Insulitis becomes evident in NOD mice at 6-8 wks. Values reflect signal over individual islets (4A) or the average signal over all of an animal's 4-10 scored islets (4B). p≦0.005 at 6-10 weeks and <0.00001 at 12-15 weeks in 4A and p=0.0002 at 12-15 weeks in 4B.

FIG. 4C is a graph illustrating the correlation between the average MFIs of CLIO signal over the islets and the fraction of infiltrated islets in individual NOD mice sorted according to age, as indicated in the legend. Values were calculated as in the legend to FIG. 3Biii.

FIG. 5A is a graph illustrating results of a comparison of image values pre- and 24 hours-post-MION treatment for BDC2.5 NOD (traingles) and E16/Nod (squares) mice. Symbols represent values for individual pancreata. P values were significantly different between the 24 hours-post-images of the two strains of mice (p=0.0001) and the pre- and 24 hours-post-images of BDC2/5/NOD mice (p<0.00001).

FIG. 5B is a pair of graphs illustrating parallel comparisons with muscle tissue; values for muscle and pancreas for sets of BDC2.5 NOD (squares) and E16/Nod (triangles) mice are plotted. Above the graphs, the delineation of ROIs for muscle (left) and pancreas (right) is illustrated.

FIG. 5C is a graph illustrating the correlation between degree of islet infiltration, calculated as in the legend to FIG. 3Biii and accumulation of MION probe.

FIGS. 6A and 6B are graphs illustrating the results of imaging of MION probe accumulation in organs of individual BDC2.5/NOD (triangles) or Eα16/NOD (squares) mice. In order to correct for mouse-to-mouse variation, values reflect the ratio of relaxation rate measured over ROIs encompassing the pancreas and muscle. Values for individual animals (6A) or average values for all individuals of each strain (6B) are plotted.

FIG. 7 illustrates the results MR imaging and analysis in a living animal. Multiple-slice, multiple-echo T2-weighted spin-echo sequences were acquired and regions-of-interest (ROIs) for analysis were defined manually on the pancreas or paraspinal muscles, as illustrated in the top image. T2 values for the individual organs were calculated by fitting a standard exponential relaxation model to the data within the ROIs. Values shown as squares (blue in original) were below background and were not included in analysis. B₀=initial magnetic field strength. Tₑ=echo time.

FIGS. 8A-F are graphs illustrating the results MR imaging and analysis of young female BDC2.5/NOD mice injected with CPA to provoke autoimmune diabetes, at the six time points shown. Each group contained 4-7 individuals. T2 values for pancreas (8A) and paraspinal muscles (8B) are shown. Vascular volume fractions (8C and 8D) and microvascular leak (8E and 8F) were estimated for each organ using formulae described herein. ** indicates P<0.01.

FIG. 9A is a set of MR images illustrating the severity of insulitis in young female BDC2.5/NOD mice imaged on Day 6 after CPA treatment and 24 hours after MNP injection. A pseudo-color was assigned to the pancreas reflecting the T2 value of the organ (indicated by white arrow in top images). In the original, the pseudocolor in the left image is bright orange and red, while the pseudocolor in the right image is light blue and indigo.

FIG. 9B is a pair of photomicrographs showing representative islet histology from the animals imaged in 9A.

FIG. 10A is a graph illustrating Mean pancreatic T2 values±SEM in female NOD mice with new-onset diabetes of less than 7 days duration (open triangles, n=9), non-diabetic female littermates of the diabetic animals (filled squares, n=7) and age-matched non-insulitic Eα16/NOD mice (filled triangles, n=13) that underwent MR imaging according to a method described herein.

FIGS. 10B and 10C are photomicrographs illustrating representative islet histology from Eα16/NOD and new-onset diabetic NOD animals, respectively.

FIGS. 11A and 11B are graphs illustrating the results of MR scanning at 13 (11A) and 20 (11B) weeks of non-diabetic female NOD mice, performed according to a method described herein. Animals were followed for the spontaneous development of diabetes until 30 weeks. Correlation between T2 value of the pancreas obtained 24 hours after MNP injection and time to diagnosis of overt diabetes is shown.

FIGS. 12A and 12B are graphs illustrating the results of MR scanning of female NOD mice with very recent onset of diabetes that were treated with either anti-CD3 or control F(ab′)₂ fragments for 5 consecutive days as described herein. MRI 24 hours post MNP injection was performed on Days 4, 8 and 18 after starting mAb immunotherapy. 12A, Results from long-term responders rendered normoglycemic following F(ab′)₂ treatment (triangles) are compared with those individuals failing to respond to therapy (i.e. anti-CD3 non-responders (squares) and control antibody non-responders (circles)). Since some individuals remained profoundly hyperglycemic for over 3 weeks in this experimental series, pancreas:muscle T2 ratio is presented to control for non-specific alterations in MRI parameters induced by these global metabolic changes. Groups represent the following number of individuals: Day 4—responders=4, non-responders=6; Day 8—responders=6, non-responders=4, control mAb treated=3; Day 18—responders=4, non-responders=6. 12B, Pancreas:muscle T2 ratio is plotted against the serum glucose measured at the time of scanning (n=33) showing no correlation between pancreatic inflammation as determined by MRI-NMP and serum glucose.

FIG. 13 is an MR image of the abdomen of a human subject who has had a previous diabetic episode 24 hours after administration of MIONs as described herein. The VVF value of the pancreas is represented using pseudocoloration, which is bright orange, red and yellow in the original. The pseudocolored area is indicated by an arrow.

### DETAILED DESCRIPTION

Type-1 diabetes is an autoimmune disease involving lymphocytic infiltration of the pancreatic islets and specific destruction of insulin-producing β cells. This process occurs over a variable number of years, eventually resulting in clinical hyperglycemia, and the diagnosis of overt diabetes. The lymphocytic infiltration continues even in patients with overt diabetes, and may last for several years into the disease course. At present this lymphocytic infiltration can only be detected by biopsy, usually at autopsy.

The ability to detect this lymphocytic infiltration through non-invasive means would have clinical benefits in at least two areas. One area would be clinical diagnostics, e.g., helping to identify patients, e.g., patients with atypical symptoms, as having type-1 rather than Type 2 diabetes, or in finding patients with pre-clinical type-1 diabetes so that they could be followed more regularly and initiate earlier treatment. Another area involves evaluation of therapeutic interventions, e.g., monitoring patients with, or at risk of, diabetes who are undergoing trial interventions to prevent, or cure, clinical type-1 diabetes. This type of trial is currently ongoing, and more trials are being planned. At the present time the only solid endpoint for these trials is clinical diabetes. This makes the trials lengthy and expensive—for example, seven years for the parenteral treatment arm of the Diabetes Prevention Trial-Type1 (DPT-1). An accurate method of following progression or regression of lymphocytic infiltration could allow for earlier discarding of ineffective treatments.

Much effort has been devoted to identifying markers that indirectly signal at least the major landmarks of T1D progression. So far, both for the NOD mouse model and for humans, the best indicator of islet inflammation appears to be serum titers of autoantibodies directed against a defined set of β-cell antigens (Eisenbarth et al., Autoimmun. Rev., 1 :139-145 (2002)). Although monitoring autoantibody titers does produce some useful information, the non-invasive methods for directly following disease progression in vivo described herein have a number of important applications for research on, diagnosis of, and treatment of T1D. For example, images of the insulitic lesion facilitates difficult diagnoses (e.g., of type-1B diabetes (Abiru et al., Diabetes Metab. Res. Rev., 18:357-366 (2002)) or of Late Autoimmune Diabetes of Adults (Naik and Palmer, Rev. Endocr. Metab. Disord., 4:233-241 (2003))), enables disease monitoring during its natural course or pursuant to treatments, e.g., immunomodulatory interventions, and provides forewarning of pancreatic tissue graft rejection. Ultimately, such images may allow the detection of early autoimmune processes in genetically at-risk individuals at the earliest stages, when therapeutic intervention is likely to be the most beneficial.

The present inventors hypothesized that modifications of the pancreatic vasculature are likely to accompany the initiation and progression of type-1 diabetes. Alterations in vascular parameters such as transient vasoconstriction, vasodilation, increased blood flow and vascular leakage are necessary preludes to inflammation and help to orchestrate the influx of diverse cell-types (Pober and Cotran, Transplantation, 50:537-544 (1990)). Changes in intra-islet vasculature precede streptozotocin-induced insulitis (Sandler and Jansson, Virchows Arch., 407:359-367 (1985); Beppu et al., Acta Endocrinol., 114:90-95 (1987); Martin et al., Diabetologia, 32:359-367 (1989); Papaccio et al., Histochemistry, 95:19-21 (1990); Papaccio and Chieffi-Baccari, Histochemistry, 97:371-374 (1992); Papaccio, Histol. Histopath., 8:751-759 (1993)), and vascular swelling and modifications of endothelial cell morphology in the para-islet region signal impending insulitis in NOD mice (Papaccio et al., Autoimmunity, 27:65-77 (1998)). In both diabetes models, and others, increased blood flow and edema ensue (Jansson and Sandler, Virchows Arch. A. Pathol. Anat. Histopathol., 410:17-21 (1986); Papaccio, Histol. Histopath., 8:751-759 (1993); Carlsson et al., Endocrinology, 139:3534-3541 (1998); Papaccio et al., J. Cell. Biochem., 86:651-664 (2002)). As described herein, such modifications are detectable using a powerful new methodology for imaging the microvasculature in rodents and humans, as described recently (Bogdanov et al., Adv. Drug Deliv. Reviews, 37:279-293 (1999); McDonald et al., Nat. Med., 9:713-725 (2003)).

As described herein, magnetic nanoparticles can be used as probes to monitor inflammation of pancreatic islets in mouse models of T1D ex and in vivo, and to image the pancreas in living human beings. This methodology permits detection of early signs of islet infiltration and allows non-invasive monitoring of lesion evolution. Thus, the methods described herein can be used to diagnose diabetes in its early stages, to predict a subject's risk of developing autoimmune diabetes, and for early monitoring of the effectiveness of an immunomodulatory therapy aimed at reversing diabetes.

MRI of magnetic nanoparticle probes (MNPs) has proven a powerful method of non-invasively visualizing vascular and cellular modifications associated with inflammation. For example, as described herein, within 24 hours after iv injection into humans or rodents, MNPs can extravasate from “leaky” vessels into the surrounding tissue and be engulfed by activated macrophages that have invaded the tissue. The present inventors hypothesized that alterations in the microvasculature accompany the leukocyte infiltration of the pancreatic islets that is the hallmark of type-1 diabetes, and that these modifications could be detected by imaging accumulation of magnetic nanoparticle probes in the pancreas.

The methods described herein can be used for non-invasively imaging inflammation of the pancreatic tissues (e.g., islets) in mammals, e.g., humans, e.g., subject with, or at risk of developing, type-1 diabetes. The experiments described herein provide proof of principle for these methods, both in murine T1D models (i.e., the clinical NOD strain and the engineered BDC2.5 strain), and in humans. The BDC2.5 T cell receptor (TCR) transgenic (tg) mouse model was used because insulitis in these animals is more synchronous and homogenous, and therefore more predictable, than it is in the standard NOD model (Katz et al., Cell, 74:1089-1100 (1993)). BDC2.5 TCR tg mice carry the rearranged TCR genes from a diabetogenic CD4⁺ T cell clone isolated from a NOD mouse and reactive to an islet β-cell antigen presented by the MHC class II molecule, A^{g7} (Haskins et al., Diabetes, 37:1444-1448 (1988); Haskins et al., Proc. Natl. Acad. Sci. USA, 86:8000-8004 (1989); Katz et al., Cell, 74 :1089-1100 (1993)). Consequently, these animals have a T cell repertoire highly skewed for the transgene-encoded, β-cell-reactive specificity (Katz et al., Cell, 74:1089-1100 (1993)). All BDC2.5 animals exhibit insulitis abruptly between 2 and 3 weeks of age, but the rate and penetrance of diabetes development varies according to the genetic background (Gonzalez et al., Immunity, 7:873-883 (1997)).

The present inventors hypothesized that administration and MR imaging of magnetic nanoparticle probes (MNP-MRI) would permit ready visualization of pancreatic microvasculature, and that detectable changes in the islet blood vessels would accompany the onset and/or progression of insulitis. Further, the inventors predicted that changes in pancreatic inflammation, as measured by MNP-MRI, would provide a direct real-time read-out of the effects of an immunointervention designed to reverse diabetes. The examples described herein demonstrate the ability of high-resolution MNP-MRI to identify and quantify the vascular volume and permeability changes associated with inflammation of the pancreas during the development of autoimmune diabetes. The methods described herein rely on measurement of the microvascular changes associated with inflammation, and are not hampered by variations in MHC alleles or autoreactive T cell specificities that limit other experimental approaches.

The methods described herein are able to non-invasively follow the initiation and progression of insulitis by MRI monitoring of MNP accumulation, in vivo in real time. In general, there appears to be a positive correlation between MNP accumulation in the pancreas and insulitis aggressivity. When applied to preclinical mouse models of autoimmune diabetes, MNP-MRI allowed non-invasive, real-time quantification of pancreatic inflammation. In the simplified BDC2.5/NOD model, microvascular changes developed explosively 3 days after cyclophosphamide (CPA) injection, just two days prior to the onset of overt diabetes Analysis of NOD mice, which more accurately model many of the features of human type-1 diabetes, confirmed the benefits of measuring nanoparticle-induced signal changes. This imaging strategy was able to differentiate between new-onset diabetics, pre-diabetic NOD littermates at risk of developing diabetes and non-insulitic Eα16/NOD mice. These results suggest, by analogy, that MRI with MNPs may be helpful in identifying human patients at immediate risk of developing autoimmune diabetes, and in differentiating between those who have type-1 and Type-2 diabetes.

Furthermore, non-invasive, real-time quantification of pancreatic inflammation was demonstrated in new-onset diabetic mice treated with a mAb specific for CD3ε. Such treatment effectively reverses hyperglycemia in a proportion of animals, and induces long-lasting protection from diabetes (Chatenoud et al., Proc Natl Acad Sci USA 91:123-127 (1994); Chatenoud et al., J Immunol 158:2947-2954 (1997); Belghith et al., Nat Med 9:1202-1208 (2003)). This therapeutic intervention is interesting because, based on the success of this therapy in rodent models, a humanized anti-CD3 mAb with decreased Fc receptor affinity is currently undergoing Phase I/II trials in patients with recent-onset type-1 diabetes, and initial results have been promising (Herold et al., N Engl J Med 346:1692-1698 (2002)).

The tolerogenic effects of treating mice with anti-CD3 mAbs seem to evolve in two distinct phases (Chatenoud et al., Nat Rev Immunol 3:123-132 (2003)): the first, occurring up to 7-8 days following initiation of anti-CD3 therapy, entails clearing the infiltrating cells from the islets; the second, long-lasting, phase is associated with active immuno-regulation and the reappearance of a lymphocytic infiltrate, but it is now non-destructive and confined to the periphery of the islets.

Non-invasive imaging in vivo allowed the identification of those animals benefiting from the immuno-modulation as early as 3 days after completing the anti-CD3 treatment course (i.e. on Day 8). Individuals that responded favorably generally experienced complete normalization of blood glucose levels within two to four weeks following treatment; hence, the beneficial effects were detectable before it was clear which animals were destined to become normoglycemic. Interestingly, imaging on Day 18 of the treatment protocol, a time when the regulated lymphocytic infiltrate is reappearing at the periphery of the islets, revealed a less marked difference in microvascular leak between mice responding favorably and those not responding to anti-CD3 therapy. This evolution may reflect a slight increase in microvascular leak associated with the accumulation of the immunoregulatory infiltrate in responding animals. A similar transient increase in microvascular leak was observed in BDC2.5/NOD mice during the development of a regulated lymphocytic islet infiltrate (Denis et al., Proc. Natl. Acad. Sci. U.S.A. (2004)).

In clinical trials, administration of a MNP to humans demonstrated that the methods can be used to image inflammation in the pancreas of living humans. As described in the Examples, below, administration of a MNP to a human being who had suffered at least one diabetic episode, e.g., a hyperglycemic episode, allowed imaging of the subject's pancreas.

Thus, described herein are non-invasive methods to monitor autoimmune inflammation in the pancreas of a living animal. The methods can be used, e.g., to detect and monitor the insulitis that accompanies the development of autoimmune diabetes, and to follow the resolution (or lack of resolution) of pancreatic inflammation after successful reversal of diabetes or insulitis with therapy.

Magnetic Nanoparticle Probes (MNPs)

The methods described herein make use of magnetic nanoparticles as contrast agents to image inflammation in the pancreas. The MNPs are used to decrease the NMR relaxation times (e.g., T1 and/or T2) of water protons in contact with a biological tissue; preferably, the MNPs enable an R2 relaxivity>50 (mM sec)⁻¹. Agents useful in the invention are particles that can extravasate from “leaky” vessels at the site of inflammation into the surrounding tissue, and be taken up and retained by macrophages in the tissue. Thus, suitable magnetic nanoparticles will have two primary properties: a sufficient plasma half life, and a sufficient macrophage avidity. A sufficient plasma half life will be at least about 5 hours in mice, and 12 hours in humans, but is more preferably 10 hours in mice and 24 hours in humans. Sufficient macrophage avidity means that the particles are taken up and retained by macrophages in sufficient quantities to allow their use as inflammation detectors. Suitable particles will be about 5 nm-200 nm in diameter; preferably, the particles are about 20-50 nm in diameter. To enhance macrophage avidity, aqueous solubility, and half-life to the particles, a coating is typically used, e.g., a coating including a starch such as dextran or modified dextran, e.g., carboxy methyl dextran, carboxy dextran, other starches including hydroxy ethyl starches, or biocompatible polymers, see, e.g., U.S. Pat. No. 5,492,814. One of skill in the art would be able to make, evaluate, and select suitable MNPs.

The MNPs used in the methods described herein are generally non-targeted; they do not need a targeting moiety that directs them to the pancreas, i.e., they lack a target specific molecule (TSM) such as is described in U.S. Pat. No. 5,492,814 that would target them to the pancreas.

In addition, MNPs are known in the art, see, e.g., superparamagnetic metal oxides as described generally in, e.g., U.S. Pat. No. 4,827,945. In preferred embodiments, the MNPs are monocrystalline superparamagnetic iron oxide particles with a dextran coating, e.g., as described in U.S. Pat. No. 5,492,814; Monocrystalline Iron Oxide Nanoparticles (MIONs)(Weissleder et al., Nat. Biotechnol., 19:316-317 (2001)), and long-circulating magnetofluorescent nanoparticles (CMFN) for fluorescence imaging (Weissleder et al., (2001), supra). MION particles contain a small, monocrystalline, superparamagnetic iron oxide core, which exhibits strong magnetic behavior and greatly improves the sensitivity of visualization. The core is surrounded by a dextran coating that serves to diminish the immunogenicity of the particles and substantially enhances their half-life in circulation. The MIONs' small size and high stability mimic the behavior of plasma proteins, rendering them highly valuable for hemodynamic studies. A further sophistication has been the generation of MION derivatives called CLIOs, which contain a Cross-Linked and aminated dextran coating around the Iron Oxide core, allowing the conjugation of “tags” such as fluorochromes or radioisotopes (e.g., an ALEXA™ fluor as described herein), and thereby permitting detection by additional imaging techniques, e.g., fluorescent imaging (Weissleder et al., Nat. Biotechnol., 19:316-317 (2001); Hogemann et al., Bioconjug. Chem., 11:941-946 (2000), and Josephson et al., Bioconjug. Chem., 10:186-191 (1999)). These imaging agents have many attractions, including their non-toxicity, non-immunogenicity, long circulation time (vascular half-lives of >10 hours) and promising performance in clinical trials. Indeed, MRI of MIONs was recently applied with great success to patients with prostate cancer, enabling visualization of small and otherwise undetectable lymph-node metastases (Harisinghani et al., N. Engl. J. Med., 348:2491-2499 (2003)).

In some embodiments, the particles are conjugated to a fluorescent moiety, e.g., as described in U.S. Pat. No. 5,492,814; Hogemann et al., Bioconjug. Chem., 11:941-946 (2000).

The particles can be provided in any suitable form, e.g., lyophilized or in a liquid, e.g., a sterile carrier that is suitable for administration in vivo, e.g., sterile saline. Lyophilized particles can be reconstituted, e.g., in normal sterile saline, or in liquid carrier. In some embodiments, the methods use Combidex® (ferumoxtran-10), a molecular imaging agent consisting of iron oxide nanoparticles, available from Advanced Magnetics, Inc., Cambridge, Mass.

The contrast agents are administered to the subject, e.g., by intravenous, intraarterial, subcutaneous, intramuscular, intraparenchymal, intracavity, topical, ocular, oral or rectal administration, with intravenous injection being preferred.

Imaging Methods

Following administration of the contrast agent, NMR imaging (e.g., spin echo, gradient echo, fast imaging, echo planar, or susceptibility imaging) is carried out; the choice of pulse sequence (inversion recovery, IR; spin echo, SE) and the values of the imaging parameters (echo time, TE; inversion time, TI; repetition time, TR) will be governed by the diagnostic information sought.

The methods include obtaining one or more images of a pancreatic tissue of the mammal, e.g., an image in which MNPs in the tissue are visible. In some embodiments, the methods include obtaining multiple (e.g., a set of) images. In some embodiments, the methods include obtaining at least one image of a pancreatic tissue prior to administration of the MNPs or derivatives thereof, and obtaining one or more additional images of the tissue after administration. In some embodiments, images are obtained at one or more of one, two, three, four, five, six, 12, 24, 36, 48 or more hours after administration. In some embodiments, images are obtained before, one hour after, and at least 24 hours after administration. Optimal dosing can be determined using methods known in the art, e.g., to produce the best images while causing the least amount of toxicity. In some embodiments, the MNPs are administered intravenously at a dose of, e.g., 0.5-5 mg Fe/kg, e.g., 1-3 mg Fe/kg, e.g., 2.6 mg Fe/kg. As one example, lyophilized MNPs can be reconstituted in normal saline and infused intravenously at a dose of 2.6 mg of iron per kilogram of body weight over a period of 15 to 30 minutes.

Suitable imaging methods include NMR imaging (MRI) and fluorescence imaging (see, e.g., Andersson-Engels et al., Phys. Med. Biol., 42:815-824 (1997)). Such methods are known in the art. In some embodiments, MRI will be performed using a conventional imaging system, e.g., a 1.5 T clinical imaging system (System 5×, General Electric Medical Systems, Milwaukee, Wis.) using a pelvic phased array coil or another appropriate imaging coil.

In general, MRI methods can include one or more of conventional spin echo (e.g., T1 and/or T2 spin echo, or T1 and/or T2 weighted spin echo (referred to as T1* and T2*)), and gradient echo (e.g., 3D gradient echo), fast imaging, echo planar, or susceptibility imaging. Alterations in vascular volume fraction (VVF) of the pancreas, caused by acute vasodilation or vasoconstriction, can be obtained by comparing the baseline T2* value with the T2* value obtained immediately after administration of MNPs. Vascular leak can be quantified by comparing the baseline T2* value with the T2* value obtained after administration of MNPs, e.g., 24 hours after administration. Thus,

VVF=ln(T2*_{pre MNP}/T2*_{immediate post MNP}), and

vascular leak=ln(T2*_{pre MNP}/T2*_{24 hrs post MNP}).

As one example, the methods can include performing at least one MR pulse sequence (and generally more than one, e.g., at least three) that includes at least a portion of the subject's pancreas, to determine values relevant to vascular volume and permeability changes associated with inflammation of the pancreas, e.g., VVF, vascular leak, T1, and/or T2* values at one or more time points. For example, one or more images of the pelvis, e.g., in slices extending from the pubic symphysis to the level of aortic bifurcation, can be obtained before and after (e.g., directly after and/or some time after) the intravenous administration of the MNPs. More than one pulse sequence can be performed at each slice level.

For example, the magnetic resonance pulse sequences can consist of one or more, e.g., all, of a T2-weighted fast spin-echo, a T2-weighted gradient-echo, and a T1-weighted two- or three-dimensional gradient-echo sequence obtained in different anatomical planes. A T2-weighted fast spin-echo can involve a repetition time of 4500 to 5500 msec, an echo time of 80 to 100 msec, a flip angle of 90 degrees, 3 excitations, a slice thickness of 3 mm, an interslice gap of 0 mm, a matrix of 256 by 256 mm, and a field of view of 20 by 30 cm. A T2-weighted gradient-echo can involve a repetition time of 300 to 400 msec, an echo time of 24 msec, a flip angle of 20 degrees, 2 excitations, a slice thickness of 3 mm, an interslice gap of 0 mm, a matrix of 160 by 256 mm, and a field of view of 22 by 30 cm. A two-dimensional T1-weighted gradient-echo can involve a repetition time of 175 msec, an echo time of 1.8 msec, a flip angle of 80 degrees, 2 excitations, a slice thickness of 4 mm, an interslice gap of 0 mm, a matrix of 128 by 256 mm, and a field of view of 22 by 30 cm. A three-dimensional T1-weighted gradient-echo can involve a repetition time of 4.5 to 5.5 msec, an echo time of 1.4 msec, a flip angle of 15 degrees, 2 excitations, a slice thickness of 5 mm, an interslice gap of 0 mm, a matrix of 256 by 256 mm, and a field of view of 24 by 32 cm.

Methods for obtaining and calculating relevant values, e.g., VVF, vascular leak, T1, and T2*, are known in the art, e.g., region of interest (ROI) analysis. See, e.g., Harisinghani et al., N. Engl. J. Med., 348(25):2491-2499 (2003). The methods can include, for example, drawing a region of interest around the pancreas and determining one or more relevant values in the absence and presence of MNPs as described herein, e.g., before and after administration of the MNPs, e.g., immediately after administration and about one or more of one, two, three, four, five, six, 12, 24, 36, or 48 hours after administration.

Subjects

The methods described herein are particularly suitable for use in subjects who are at risk of developing type-1 diabetes. For example, such subjects include those who have a genetic predisposition to develop type-1 diabetes, e.g., subjects with one (or more) first degree relative(s) who has (have) the disorder, e.g., a sister, brother, mother, father, son, or daughter. Subjects at risk can also be identified, e.g., by the presence of immune markers in the blood such as antibodies against insulin, islets, or the enzymes glutamic acid decarboxylase (GAD) and IA2 (also known as ICA512). These markers can be detected using methods known in the art, e.g., blood tests for autoantibodies to components of the insulin-producing islet cells and to insulin itself. Alternatively, subjects can be identified using a blood test for specific genes that may make individuals more susceptible to type-1 diabetes, e.g., in the IDDM1 locus, the presence of alleles HLA-DQB1 and HLA-DRB1, and alleles such as HLA-DR3 or HLA-DR4 in Caucasians, HLA-DR7 in persons of African descent, and HLA-DR9 in Japanese. See, e.g., OMIM Entry No. +222100.

Glucose tolerance tests can also be used, e.g., oral glucose tolerance tests (OGTT). Oral glucose testing is typically performed after a high carbohydrate diet for three days followed by a 10 to 14 hour fast prior to the start of the test. To begin, blood is drawn for a fasting glucose level, and then a 75 gram oral glucose load is administered, e.g., a beverage containing 75 grams glucose. Blood is then drawn, e.g., every half hour or every hour for 2 hours, or just at 2 hours. Plasma glucose levels at or above 200 mg/dl, or a fasting glucose of 126 or above is diagnostic of diabetes. A fasting plasma glucose of 100-125 or 2 hour plasma glucose of 140-199 are significant for “pre-diabetes.” Concurrent measurement of insulin and/or C-peptide levels can also be used to help determine if the diabetes is from increased insulin resistance or decreased insulin production.

An exemplary method is a measurement of the amount of insulin produced in response to an intravenous injection of glucose. Normally, there is a biphasic insulin response to IV glucose. The first phase can be lost before abnormalities are seen on a 2 hour 75 gm oral glucose tolerance test. People who have lost the first phase insulin response may not yet meet diagnostic criteria for diabetes, but are at very high risk for developing diabetes.

Finally, epidemiologic studies have suggested that a number of environmental factors might influence the development of diabetes. These include viral infections (especially a history of enterovirus infections, particularly with Coxsackie B), ever life stressors, and several dietary factors.

Diagnosis and Prognosis

Methods described herein can be used to identify and track pancreatic inflammation during the unfolding of autoimmune diabetes, to establish a diagnosis of T1D in the absence of standard indications in atypical patients, and to predict whether and when overt diabetes will develop. Generally, MR images of the pancreas are obtained as described herein, and at least one relevant value associated with vascular permeability changes in the pancreas, e.g., vascular volume (i.e., VVF) and/or vascular leak, is determined. The relevant values can then be compared with a reference value, e.g., a reference that is associated with the presence or absence of autoimmune diabetes. For example, the reference can be a threshold value, and a relevant value for the subject that is above the reference threshold indicates that the subject has diabetes. As another example, the reference value can be a range, and a relevant value for the subject that is above, in, or below the range indicates whether the subject is likely to develop diabetes in the future.

Reference values can be established using the methods described herein, e.g., in subjects who are known to be healthy, or in the early stages of autoimmune diabetes. Methods used to determine appropriate reference values for prostate cancer, e.g., as described in Harisinghani et al., PLoS Med. 2004 December;1(3):e66, can be adapted to determine appropriate reference values for a method described herein.

Additional images of the same subject can be obtained at later time points, and the relevant values obtained using those images can be compared with earlier images to evaluate the development or progression of inflammation in the pancreas.

Evaluation of Therapeutic Interventions

The methods described herein can also be used to evaluate therapeutic interventions, e.g., in clinical trials. For example, the methods can be used to determine whether a treatment will prevent or delay the development of type-1 diabetes, or halt its progression in recent onset diabetic subjects, or reverse insulitis. Generally, a set of first MR images of the pancreas are obtained as described herein, and at least one initial relevant value associated with vascular permeability changes in the pancreas, e.g., vascular volume (i.e., VVF) and/or vascular leak, is determined. The subject is then administered a test intervention, e.g., a potential treatment or preventive for diabetes, e.g., a test compound, alteration in diet and/or exercise, cell transplant therapy, or other intervention that is known or suspected to have an effect on the development, regression or progression of autoimmune diabetes. Additional images are then obtained at at least one later time point, and the relevant values obtained using those images are compared to the initial images to evaluate the progression of inflammation in the pancreas, and thereby evaluate the therapeutic intervention. As one example, a change, e.g., a decrease in VVF or vascular leak would indicate that the therapeutic intervention is effective in treating or delaying the development, regression or progression of autoimmune diabetes.

Insulitis

Insulitis is defined as a histologic change in the islets of Langerhans, characterized by edema and the infiltration of white blood cells. Insulitis can include acute insulitis, chronic insulitis, and lymphocytic insulitis, and can be caused by, e.g., environmental toxicity, or viral or bacterial infection. Insulitis is sometimes the initial lesion of type-1 diabetes mellitus, but does not always progress to full type-1 diabetes mellitus.

Diabetes Mellitus

A diagnosis of type-1 diabetes mellitus can be made, e.g., on the basis of symptom history confirmed by a blood or plasma glucose level greater than 200 mg/dl, with the presence of glucosuria and/or ketonuria. Other symptoms representative of autoimmune diabetes are polyuria, polydipsia, weight loss with normal or even increased food intake, fatigue, and blurred vision, commonly present 4 to 12 weeks before the symptoms are noticed. Before clinical onset of type-1 diabetes mellitus, diagnosis may be possible with serologic methods, e.g., complemented by beta cell function tests.

A positive effect on a parameter associated with diabetes can be one or more of the following: (1) decreasing plasma glucose levels and urine glucose excretion to eliminate polyuria, polydipsia, polyphagia, caloric loss, and adverse effects such as blurred vision from lens swelling and susceptibility to infection, particularly vaginitis in women, (2) abolishing ketosis, (3) inducing positive nitrogen balance to restore lean body mass and physical capability and to maintain normal growth, development, and life functioning, (4) preventing or greatly minimizing the late complications of diabetes, i.e., retinopathy with potential loss of vision, nephropathy leading to end stage renal disease (ESRD), and neuropathy with risk of foot ulcers, amputation, Charcot joints, sexual dysfunction, potentially disabling dysfunction of the stomach, bowel, and bladder, atherosclerotic cardiovascular, peripheral vascular, and cerebrovascular disease. A negative effect on a parameter would, of course, be the reverse. The current American Diabetes Association standards of care include (1) maintaining preprandial capillary whole blood glucose levels at 80 to 120 mg/dl, bedtime blood glucose levels at 100 to 140 mg/dl, and postprandial peak blood glucose levels at less than 180 mg/dl, and (2) maintaining an HbA1c of less than 7.0% (relative to a nondiabetic DCCT range of approximately 4.0% to 6.0%).

A number of candidate treatments for diabetes are known in the art and in development; the methods described herein can be used to determine which are effective in reducing pancreatic inflammation. Such candidate treatments can include administration of therapeutic compounds, including new potential therapeutic compounds, diet and/or exercise regimes, or other therapeutic modalities. New or non-conventional methods of administering known therapeutic compounds can also be evaluated by these methods, e.g., oral or nasal administration of insulin.

The invention is further described in the following examples, which do not limit the scope of the invention described in the claims.

### EXAMPLES

### Example 1

### Ex Vivo Visualization of Microvascular Leakage in the BDC2.5 Model

Since an increase in the permeability of small blood vessels is a known antecedent to inflammation, the experiments described herein first sought to image leakage from microvessels associated with the pancreatic islets of BDC2.5 TCR tg mice. Work on other systems has established that shortly after intravenous (iv) injection, MION/CLIO probes remain largely within the blood vessels, permitting visualization of the vascular bed; but that by 24 hours post-administration, they have leaked into the surrounding tissue and, thereby, signal changes in vascular permeability (Weissleder et al., Radiology, 175:489-493 (1990); Weissleder et al., Radiology, 191:225-230 (1994)).

NOD/Lt, Eα16/NOD, NOD-RAG^{0/0}, BDC2.5/NOD, BDC2.5/B6.H-2^{g7/g7}, and BDC2.5/NOD-RAG^{0/0} mice were bred in our animal facility under specific-pathogen-free conditions. Diabetes was monitored by measuring glucose in the urine (Diastix, Bayer Co., Elkhart, Ind.). Two consecutive positive measurements were considered indicative of diabetes, which was confirmed by blood glucose measurements (Glucometer Elite, Bayer Co., Mishawaka, Ind.). Negative control animals, devoid of insulitis, were routinely NOD-RAG^{0/0} or Eα16/NOD mice.

To facilitate rapid evaluation and optimization of imaging protocols in the BDC2.5 context, ex vivo microscopic visualization of fluorescently labeled MION derivatives in excised pancreata was initially employed. For the confocal microscopy studies, CLIO (Hogemann et al., Bioconjug. Chem., 11:941-946 (2000); Josephson et al., Bioconjug. Chem., 10:186-191 (1999)), a fluorescent modification of MION that allows its detection by optical methods, was used. CLIO consists of a core of superparamagnetic iron oxide and a crosslinked dextran coating with amino groups to which Alexa-488 fluorochrome (Molecular Probes, Inc., Eugene, Oreg.) is attached.

The standard protocol was to inject animals iv with CLIO-Alexa-488 (green) or, as a negative control, with phosphate-buffered saline (PBS); to inject them 24 hours later with Dithizone (DTZ) or Hoechst (HST) dyes to stain the islets (red) or nuclei (blue), respectively; and, 5 minutes later, to sacrifice them, remove their pancreas and visualize the fluorescent signals by confocal microscopy. Diphenylthiocarbazone (Dithizone, DTZ) (Sigma, St. Louis, Mo.) acts as a chelator and labels islets red due to their high zinc content. DTZ can be detected by confocal microscopy due to its autofluorescence properties in the red channel. Hoechst (list) 33342 dye (Calbiochem, San Diego, Calif.) was used to demarcate nuclei.

Routinely, animals were iv-injected with 10 mg/kg of CLIO-Alexa-488, and pancreata were excised for imaging after 5 minutes or 24 hours. DTZ and HST counterstaining, perfusion and fixation procedures varied according to the experiment, as detailed herein. The whole pancreas was placed on a slide, covered with a No.1 cover-glass (Corning Inc., Acton, Mass.) and kept moist with PBS while imaged with an inverted laser-scanning microscope (Zeiss LSM 410) (Carl Zeiss, Thornwood, N.Y.). Areas containing islets were identified based on their DTZ staining, and an average of 4-10 islets were imaged for each experimental animal. An ArKr 488/568/647 laser provided the excitation wavelengths that allowed simultaneous detection of red and green images, which were acquired with the 10× objective and an adjusted pinhole to obtain an optical section thickness of 50 μm. For the closer inspection of probe localization, cell nuclei were revealed by staining with Hst. list was excited with the UV laser (Ar 364) and images were acquired with the 40× oil immersion objective and an optical section thickness of 8 μm. Images were acquired using the LSM 410 software package from Carl Zeiss and analyzed with custom-developed LAB-VIEW-based image-analysis software. Regions of interest (ROIs) were drawn around either islets defined according to DTZ staining or exocrine pancreas, and the MFI of the long-circulating magnetofluorescent nanoparticles (CMFN)/Alexa-488 green fluorescence was measured based on values of individual pixel intensities. An average of 4-10 islets were measured for each animal.

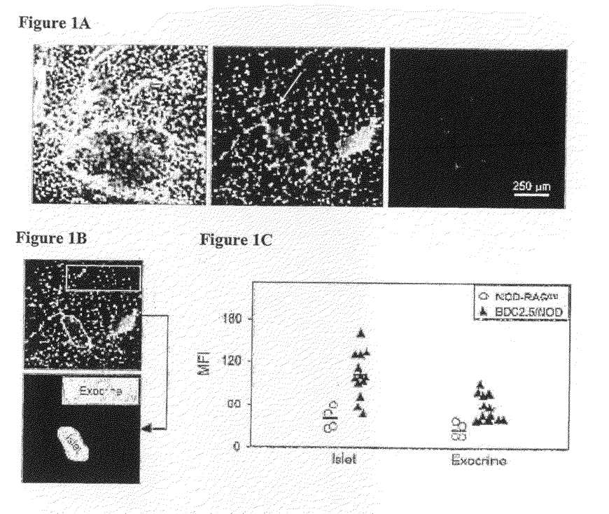

A clear augmentation in the extent of leakage from the islet microvasculature, reflected as an accumulation of the CLIO probe, was evident on the pancreas images derived from insulitic 4-week-old BDC2.5/NOD mice (FIG. 1a). Strong green fluorescence surrounded the red-stained islets of pancreata from these animals (left panel), in contrast to the weak green fluorescence in islet regions of pancreata from age-matched, non-insulitic NOD-RAG^{0/0} controls (center panel), or the absence of autofluorescence in analogous regions of control, PBS-injected animals (right panel). The weak fluorescence associated with normal islets can probably be attributed to a slight amount of leakage from the fenestrated islet microvasculature (Henderson et al., Q. J. Exp. Physiol., 70:347-356 (1985)).

CLIO fluorescence was quantitated in the peri- and extra-islet regions of the pancreas images using home-built software (panel 1b; detailed in Methods). All data described herein were analyzed using a two-tailed and two-sample unequal variance Student's t-test. Values of P≦0.05 were considered significant. These analyses revealed statistically significant accumulation of the probe in the insulitic pancreata of BDC2.5/NOD mice, most strikingly in regions immediately surrounding the islets (p=0.0003), but also spilling over into exocrine regions (p=0.003) (panel 1c).

The mechanisms underlying CLIO probe accumulation in insulitic pancreata were explored by performing a perfusion experiment, flushing PBS then formalin through the left ventricle of the heart just before removal of the pancreas. Briefly, 4-week-old female BDC2.5/NOD and Eα16/NOD mice were injected with CLIO-Alexa-488 as described above. 24 hours later, animals were sub-lethally anesthetized and iv injected with 25 mg/kg Hst to stain nuclei, and after 5 minutes, were perfused through the left ventricle with PBS/Heparin followed with 5 ml 10% neutral-buffered formalin to fix the pancreas. This process removed all of the blood as well as all of the remaining circulating CLIO. The pancreas was immediately removed and further fixed overnight in 10% neutral-buffered formalin. Subsequently, it was placed in glycerol-based mounting medium, where it was kept for approximately one week until it was transparent enough to visualize by confocal microscopy.

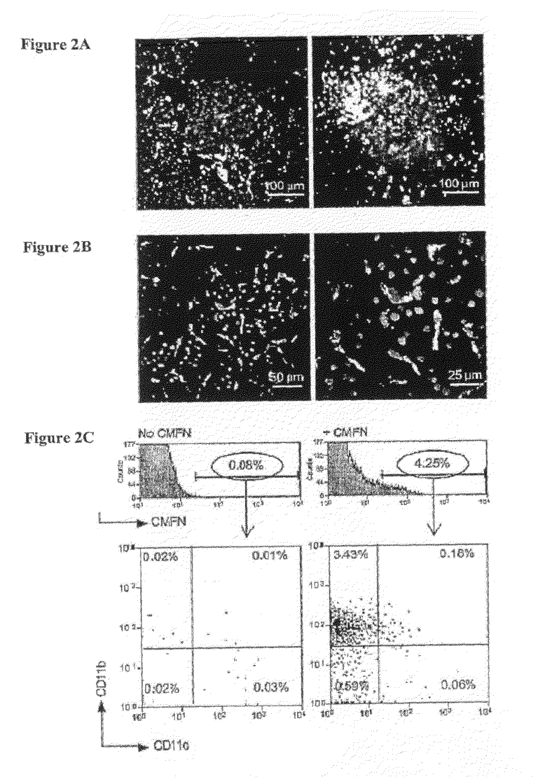

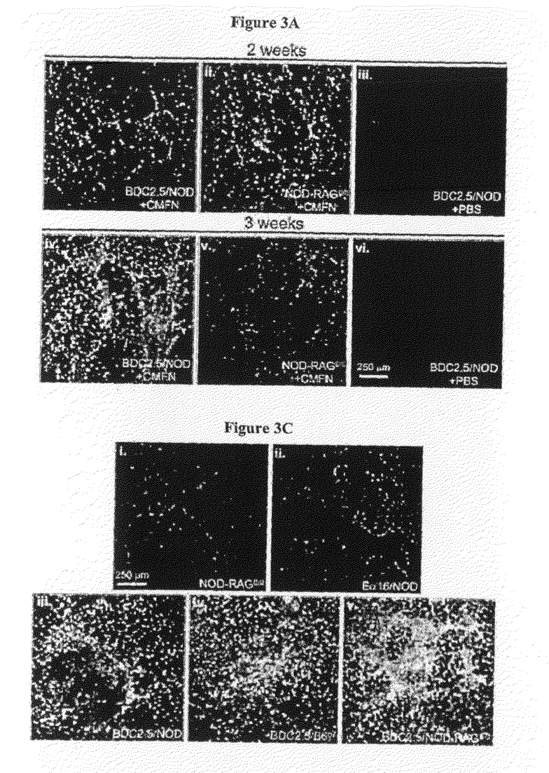

The increase in green fluorescence in insulitic versus non-insulitic pancreata was just as evident after perfusion (FIG. 2a and data not shown), suggesting that, once extravasated into the interstitium, the CLIO probe was rapidly taken up by cells in the neighborhood. These cells could be visualized via higher magnification microscopy of pancreas tissue wherein cell nuclei had been counter-stained blue with Hst. The distribution of the cells suggested that they were participants in the leukocytic infiltrate, clumped in close association with the islets (FIGS. 2a and b). The cells' morphology was consistent with their being macrophages or dendritic cells (FIG. 2b), as has been reported in past studies on other systems (Moore et al., Radiology, 214:568-574 (2000)).

Cytofluorimetric analysis was performed to confirm these results. A group of three 4-week-old female BDC2.5/NOD mice was used per sample. One group was left injected while the other was iv injected with 10 mg/kg CLIO-Alexa-488 and was otherwise treated as above, except no counterstain or fixative was administered. Animals were perfused with 15 ml PBS containing 1 U/ml heparin (Sigma-Aldrich, St. Louis, Mo.), and their pancreata were excised, teased, and digested with collagenase P (Roche Molecular Biochemicals, Indianapolis, Ind.) at 37° C. for 20 minutes. Digested material was pipetted vigorously and filtered in cold wash buffer containing 5 mM EDTA. Bulk organ suspensions were stained for multicolor flow cytometry with the following antibodies: CD3-FITC (17A2) and CD11c-PE (HL-3) purchased from BD Biosciences (San Diego, Calif.), CD45R-PE Texas Red (RA3-6B2) from Caltag (Burlingame, Calif.) and CD11b-biotin (M1/70) from eBioscience (San Diego, Calif.) detected with Streptavidin-APC (BD Pharmingen). Samples were acquired with a DakoCytomation MoFlo® High-Performance Cell Sorter (Fort Collins, Colo.) and analyzed using the Summit software (DakoCytomation). This analysis confirmed that the CLIO-labeled cells had a cell-surface phenotype (CD11b⁺11c⁻) characteristic of macrophages (FIG. 2c).

These data establish the utility of the CLIO-Alexa-488 probe for exploring the microvasculature of the pancreatic islets, and demonstrate that insulitis is accompanied by an increase in probe extravasation/accumulation. This concentration in extra-vessel regions probably results both from an increase in microvascular permeability, promoting probe leakage, and an influx of phagocytic macrophages, permitting probe up-take and thereby inhibiting its diffusion.

### Example 2

### Relationship Between CLIO Probe Accumulation and the Aggressiveness of the Insulitic Lesion

An obvious question that arises is how the accumulation of extravasated CLIO probe correlates with insulitis aggressivity, both in a temporal and a compositional sense. How does the fluorescent signal intensity change with the unfolding of the lesion? How is it influenced by the lesion's leukocyte participants? Experiments designed to answer these questions exploited BDC2.5/NOD mice of various ages or with diverse genetic lesions. The protocol was the standard one described above, permitting confocal imaging of green-stained vascular probe and red-stained islets in pancreata 24 hours after iv injection of CLIO probe. (No perfusion was performed in order to maximize the signal from extravasated probe.)

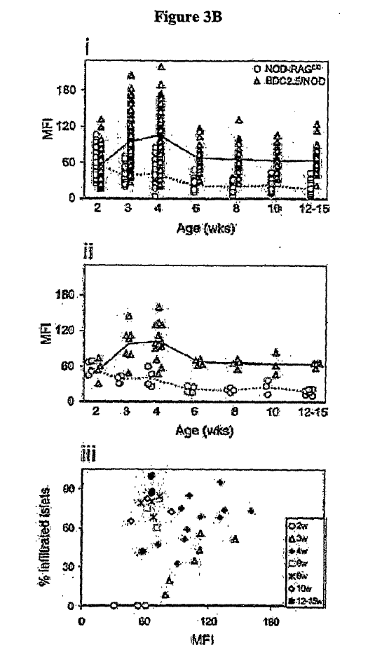

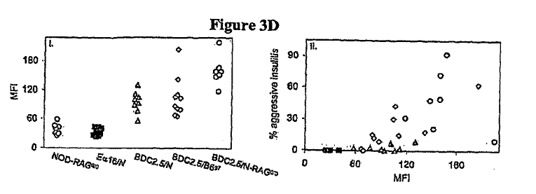

Insulitis develops abruptly and synchronously between two and three weeks of age in BDC2.5/NOD mice (Luhder et al., J. Exp. Med., 187:427-432 (1998)). This behavior was nicely “captured” by the CLIO probe: an intense green signal surrounded the islets in images from 3-, but not 2-, week-old animals (FIG. 3ai vs. 3aiv). Such a difference was not observed on images from age-matched, non-insulitic NOD-RAG^{0/0} animals (FIG. 3aii vs. 3av), nor in the absence of probe (FIG. 3aiii vs. 3avi).

Next, a quantitative kinetic analysis (FIG. 3B) was performed, from which several salient points emerged. To histologically evaluate insulitis, mouse pancreata were collected and fixed overnight in 10% neutral-buffered formalin (Sigma Diagnostics, St. Louis, Mo.). Thin sections of paraffin-embedded pancreata were examined for the presence of insulitis after hematoxylin-eosin staining. Multiple sections were taken from at least three different levels, selected as being representative of the whole organ. “Insulitis” refers to lesions with a clear and often extensive islet infiltrate exhibiting direct lymphocyte-β cell contact, but with an obvious demarcation of the infiltrate and relatively healthy β cells. “Aggressive insulitis” refers to an extensive infiltrate, where lymphoid cells invade the entire islet, intermingling with endocrine cells, with extensive signs of β cell damage.

Changes in CLIO extravasation/accumulation did occur over time, and this was true whether the mean fluorescence intensity (MFI) surrounding individual islets or the average peri-islet MFI for individual animals was plotted (FIGS. 3Bi and ii). In the case of non-insulitic pancreata from NOD-RAG^{0/0} mice, islet and animal MFI values dropped steadily from two to fifteen weeks of age, probably reflecting maturation of the vascular architecture, closing remaining gaps between endothelial cells and establishing tight junctions (Guo et al., J. Immunol., 166:1073-1086 (2001); Bellhorn et al., Invest. Ophthalmol. Vis. Sci., 21:282-290 (1981)). For insulitic pancreata from BDC2.5/NOD mice, islet and animal MFIs were minimal at 2 weeks of age, indistinguishable from those of NOD-RAG^{0/0} controls of the same age. There was an abrupt increase in BDC2.5/NOD MFIs at 3 weeks, which maximized at 4 weeks. By 6 weeks of age, MFIs had fallen to values essentially indistinguishable from those of 2-week-old pre-insulitic animals, and these remained steady out to 15 weeks. Despite the lower BDC2.5/NOD MFI values after 6 weeks of age, they remained significantly above the analogous values derived from age-matched, non-insulitic NOD-RAG^{0/0} controls. At each time-point, there was a great deal of scatter in MFI values, particularly for individual islet MFIs from BDC2.5/NOD animals; there is also a great variation in the degree of infiltration of individual islets in these animals (data not shown). There turned out not to be a simple linear relationship between the degree of insulitis and level of fluorescence intensity (FIG. 3Biii). In younger mice (<4 weeks), the MFI did increase along with infiltration, but once the islets became heavily infiltrated (>4 weeks), the MFI remained stable or even declined.

Thus, the longitudinal study of insulitis evolution in BDC2.5/NOD mice generated a CLIO accumulation curve with an unexpected shape: an abrupt increase between 2 and 3 weeks of age, and an abrupt decrease between 4 and 6 weeks, followed by little change out to 15 weeks. The punctual increase between 2 and 3 weeks of age was exactly as expected, a nice reflection of the punctual initiation of insulitis during this precise time-window (Hoglund et al., J. Exp. Med., 189:331-339 (1999)). The sudden decrease between 4 and 6 weeks is more difficult to explain. CLIO/MION accumulation at sites of inflammation signals at least two processes: an increase in probe extravasation, which could be due to an augmented vessel leakage, blood flow and/or vessel density; an increase in probe uptake, which might signal more or different phagocytic cells in the vicinity. Any of these parameters might change through evolution of the insulitic lesion in BDC2.5 mice. Indeed, 3D reconstruction of confocal images of pancreata from BDC2.5 animals perfused with PBS/Heparin then with CLIO probe plus fixative and immediately dissected suggests a decrease in peri-islet vessel diameter and density as disease unfolds (M. C. Denis, unpublished observations). There have also been reports of changes in macrophages and/or dendritic cell populations through the evolution of insulitis in NOD mice, in particular in the 3-7 week time-window (Jansen et al., Diabetes, 43:667-675 (1994); Shinomiya et al., Pancreas, 20:290-296 (2000); Murata et al., Eur. J. Immunol., 33:1001-1011 (2003)).

Genetic variants of the NOD and BDC2.5/NOD models permit one to explore the influence of lesion composition on CLIO probe extravasation/accumulation. NOD-RAG^{0/0} mice lack α:β T and B lymphocytes, and consequently do not develop insulitis (Gonzalez et al., Immunity, 7:873-883 (1997)); Eα16/NOD animals have seemingly normal α:β T and B lymphocyte compartments, but are protected from insulitis, likely due to a greater number or activity of regulatory T cells (Böhme et al., Science, 249:293-295 (1990)). These two NOD variants are similar, then, in being non-insulitic, but differ in their repertoire of lymphocytes. In no case was significant extravasation/accumulation of the CLIO probe observed in the pancreas of 4 week-old individuals, reflecting an intact microvasculature (pictured in FIGS. 3ci and 3cii; quantitated in FIG. 3di).

The appearance of the islet infiltrates in young BDC2.5 TCR tg mice differs according to the genetic background (Gonzalez et al., Immunity, 7:873-883 (1997)). Three- to 4-week-old BDC2.5/NOD animals have a very dense, but innocuous-appearing, lesion that circumscribes the β-cell mass. Age-matched BDC2.5/B6.H-2^{g7/g7} and BDC2.5/NOD-RAG^{0/0} animals have more aggressive lesions, with activated leukocytes, dying β-cells and heavy intermingling between the two, the latter strain being the most extreme (Gonzalez et al., Nat. Immunol., 2:1117-1125 (2001)). In all three cases, pancreas images revealed strong extravasation/accumulation of the CLIO probe, indicative of microvascular leakage (pictured in FIG. 3ciii-v, quantitated in FIG. 3di). It was hypothesized that the scatter in MFI values for mice of the same strain might not reflect individual variation in the progression of insulitis. A plot of the fraction of islets with an aggressive infiltrate versus average peri-islet MFI revealed that this was, indeed, the case (FIG. 3dii).

Clearly, then, the CLIO probe was able to portray the degree of insulitis aggressivity—both during unfolding of the BDC2.5/NOD lesion through time, and with the variably destructive lesions of NOD and BDC2.5 variants with different mutations or on different genetic backgrounds.

### Example 3

### Ex Vivo Visualization of Microvascular Leakage in the NOD Model

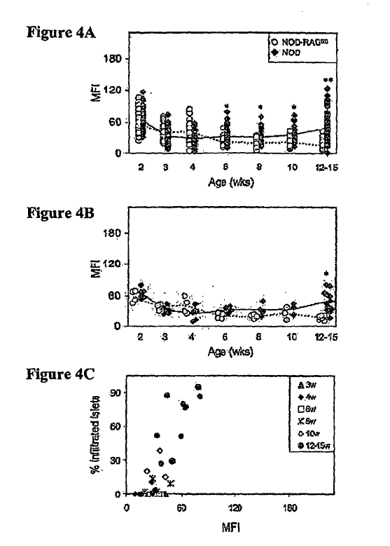

Insulitis is less synchronous and florid in NOD mice, a more complex type-1 diabetes model: routinely, it becomes evident at 6-8 weeks of age, but initiates and progresses with variable rapidity and severity in different individuals. Hence, the CLIO-Alexa-488 probe was used to assay microvascular leakage in a number of NOD mice at ages ranging from 2 to 15 weeks employing the same protocol that had been used with BDC2.5/NOD TCR tg animals. Age-matched NOD-Rag^{0/0} animals devoid of insulitis were tested in parallel as negative-controls. MFI values reflecting probe extravasation/accumulation in the peri-islet region were extracted from confocal images and plotted. The time-course curves for NOD mice (FIGS. 4a and b) were clearly different from those generated using BDC2.5 animals (FIGS. 3Bi and ii). Whether one considers individual islets (FIG. 4a) or individual animals (FIG. 4b), the signals surrounding the islets of NOD mice were not clearly different from those associated with NOD-RAG^{0/0} islets until the latest, 12-15 week, time-point (FIG. 4a: p<0.00001; 4b: p=0.0002). Also distinguishing the data-sets from the two strains was the relationship between probe accumulation (MFI) and insulitis aggression (% infiltrated islets): close to linear for NOD mice (FIG. 4c) and a much more complex curve for BDC2.5/NOD animals, suggestive of evolving vascular parameters (FIG. 3Biii). However, this distinction may simply reflect less “mature” insulitic lesions in 12-15 week-old NOD mice than in BDC2.5/NOD animals of the same age.

Thus, the CLIO-Alexa-488 probe also permits monitoring of insulitis progression in standard NOD mice, although the signal differential using this protocol may be less impressive than with the BDC2.5 TCR tg model.

### Example 4

### In Vivo Imaging of Microvascular Leakage in Mice

This Example describes methods for using in vivo imaging of microvascular leakage as a non-invasive measure of insulitis progression.

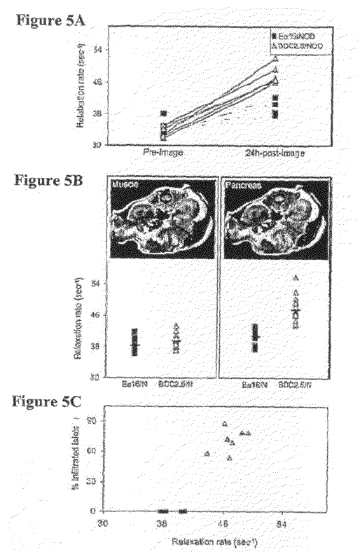

Because the probe was intended to be visualized by MRI, and therefore relied only on its magnetic properties, MION (rather than CLIO-Alexa-488) was used as an imaging agent; for all mouse MRI studies, the previously described MION-47 (Weissleder et al., Radiology, 175:489-493 (1990); Shen et al., Magn. Reson. Med., 29:599-604 (1993)) was used. Four-week-old BDC2.5/NOD or age-matched non-insulitic negative-control Eα16/NOD mice were anesthetized and MR “pre-imaged,” were iv injected with 40 mg/kg MION-47, and 24 hrs later were anesthetized and MR “post-imaged.” A “region of interest” (ROI) was drawn around the pancreas (e.g., as illustrated in the upper panel of FIG. 5b) and the signal in a 1 mm slice was quantitated as the reciprocal of the relaxation rate.

T2 measurements in mice were performed with an 8.5 Tesla micro-imaging system (DRX-360, Bruker BioSpin MRI, Inc., Karlsruhe, Germany). 24 hours after iv injection of 40 mg/kg MION-47, mice were anesthetized by inhalation of 1-2% isoflurane (Forane, Abbott Laboratories, North Chicago, Ill.) and placed in a bird-cage radio-frequency coil with an inner diameter of 20 mm. The imaging protocol included multiple-slice, multiple-echo T2-weighted spin-echo sequences with respiratory gating and TR/TEs=2000/6.6 to 66 msec, FOV=2.5-3.0 cm, matrix size 128×128, slice thickness=1 mm interleaved, bandwidth=10 kHz and number of excitations (NEX)=4. A total of 11 sequential axial images were obtained on the entire pancreas. Actual imaging time was approximately 17 minutes, in part due to respiratory gating. Imaging analysis was performed using CMIR-IMAGE custom-designed software developed in Interactive Data Language (Research Systems Inc, Boulder, Colo.). Regions of interest (ROIs) for analysis were defined manually, and were within the boundary in-plane of the visualized pancreas on three consecutive slices to ensure no contamination of signal secondary to volume averaging with adjacent organs. Only the center slice was used for analysis. Relaxation rates were acquired by performing fits of a standard exponential relaxation model to the data on a pixel-by-pixel basis within the ROIs.

Pre-imaging T2* values for pancreata from BDC2.5/NOD and Eα16/NOD mice were indistinguishable, as were pre- and 24 hours-post-imaging figures for pancreata from Ea16/NOD animals (FIG. 5a). However, 24 hours-post-imaging values for the two strains were significantly different (p=0.0001), and pre- and 24 hours-post-imaging figures for BDC2.5 pancreata differed significantly as well (p<0.00001) (FIG. 5a). These divergences were specific to the pancreas as they were not observed in other tissues, such as muscle (FIG. 5b).

In general, those animals with the highest pancreas values exhibited the most extensive insulitis, as scored histologically (FIG. 5c).

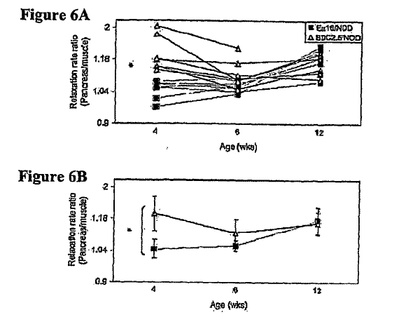

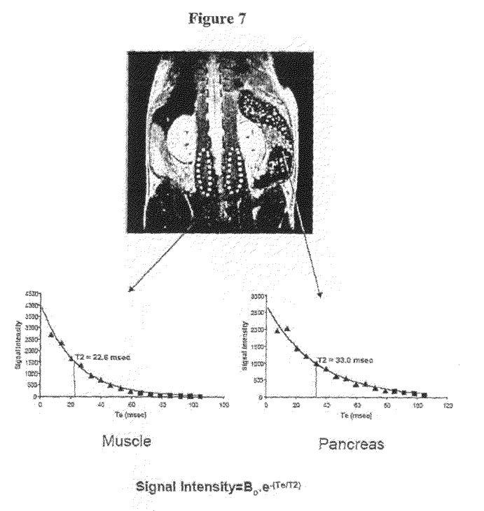

Next, the techniques described herein were used to follow the progression of insulitis in individual animals through time (FIG. 6). Much as had been seen on the population level, by visualizing peri-islet accumulation of the CLIO-Alexa-488 probe in pancreata ex vivo, there was a significant concentration of MION in insulitic pancreata of 4-week-old BDC2.5/NOD mice, vis a vis in the non-insulitic organ of age-matched Eα16/NOD controls (p=0.009). This difference had disappeared by 6 weeks of age and values remained essentially stable thereafter.

These results demonstrate that the evolution of insulitis in BDC2.5 TCR tg mice can be monitored non-invasively in vivo in real time by measuring leakage of the microvasculature via MRI of a MION probe.

### Example 5

### Evolution of Pancreatic Inflammation During the Development of Cyclophosphamide-Induced Diabetes in BDC2.5/NOD Mice

The overarching goal of these experiments was to provide proof-of-principle preclinical data on mouse models of type-1 diabetes to guide the safe and successful application of an in vivo MION imaging technique to patients with, or at risk of, autoimmune diabetes. A logical first step was to track the inflammatory signals arising from the pancreas during disease unfolding. As it is impossible to predict the exact age at which diabetes will spontaneously appear in a given NOD mouse, the simpler BDC2.5 TCR Tg model was initially employed. These mice have a T cell repertoire highly skewed towards β-cell-reactivity. All animals exhibit insulitis abruptly between 2 and 3 weeks of age, but the rate and penetrance of diabetes development varies according to the genetic background, a phenomenon now known to reflect immunoregulation (Gonzalez et al., Nat. Immunol., 2:1117-1125 (2001)). Treatment of BDC2.5/NOD mice with cyclophosphamide (CPA) destabilizes the immunoregulatory balance and induces autoimmune diabetes in 100% of animals between 4 and 10 days following injection (Andre-Schmutz et at, Eur. J. Immunol., 29:245-255 (1999)). Such rapid, highly reproducible kinetics allow a detailed temporal analysis of inflammatory changes occurring within the pancreas during the development of autoimmune diabetes.

Diabetes was provoked in 6-8 week old female BDC2.5/NOD mice by intraperitoneal injection of CPA (200 mg/kg) using established protocols (Andre-Schmutz et al., Eur. J. Immunol., 29:245-255 (1999)). To allow survival of BDC2.5/NOD mice for analysis 35 days after CPA treatment, exogenous insulin was provided by subcutaneous insulin-releasing implants (LinBit, LinShin, Canada).

MRI was performed at various time-points thereafter, as follows. A 4.7 Tesla micro-imaging system (Bruker Pharmascan, Karlsruhe, Germany) was used to perform MRI. Mice were anesthetized by inhalation of isoflurane and were placed in a bird-cage radio-frequency coil with an inner diameter of 36 mm. The imaging protocol included multiple-slice, multiple-echo spin-echo sequences (TR=2000 msec, TE=6.5 to 195 msec, FOV=3.5 cm, matrix size 128×128, slice thickness=0.6 mm interleaved, number of excitations=4). A total of 16 sequential coronal images were obtained to cover the entire pancreatic region.

MIONs (monocrystalline iron oxide nanoparticles; MION-47) were injected at 20 mg/kg Fe to allow quantification of microvascular changes associated with pancreatic inflammation (Bremer et al., Radiology, 226:214-220 (2003)). MION-47 (Center for Molecular Imaging Research, Massachusetts General Hospital, Harvard Medical School, Boston) had a size of 22 nm and an R2 relaxivity of 69 mMsec⁻¹ at 37° (0.47 T). Mice underwent scanning immediately before and after MION-47 injection, and a third scan was performed 24 hours after MION-47 administration to quantify microvascular leakiness.

Image analysis was performed using CMIR-Image custom software developed in Interactive Data Language (Research Systems Inc, USA). Regions-of-interest (ROIs) for analysis were defined manually on the pancreas or para-spinal muscles on three consecutive slices. To ensure that there were no volume-averaging effects with adjacent organs on calculated T2 values, the ROIs were propagated to adjacent slices, and were modified as needed such that the windows on original and adjacent slices contained only the tissue of interest. T2 values for individual organs were calculated by fitting a standard exponential relaxation model to the data averaged over the entire ROIs on each slice. Values less than background were not included in analysis (FIG. 7). The mean T2 value for the three consecutive slices was then calculated to determine the value for each organ. For some experiments vascular volume fraction (VVF) [VVF=ln(T2_{pre MION}/T2_{immediate post MION})] and vascular leak [Vascular leak=ln(T2_{pre MION}/T2_{24 hrs post MION})] were calculated as described previously (Bremer et al., Radiology, 226:214-220 (2003)).

Routinely, animals were anesthetized and a baseline MR image obtained. Without removing them from the MR scanner, MION was injected intravenously, and a second image was obtained immediately in order to yield vascular volume data from the blood-borne nanoparticles. 24 hours later, mice underwent a third scan to provide information about microvascular permeability. A region of interest (ROI) was drawn around the pancreas (FIG. 7), and the T2 value of the organ was calculated, the presence of nanoparticles within an organ decreasing its T2 value.

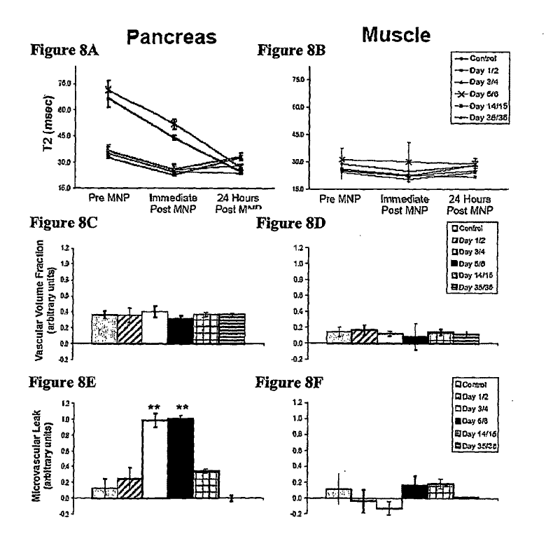

It was striking how well the MRI observations, made non-invasively on live animals, reflected the histological changes that accompany CPA-triggered diabetes in the BDC2.5/NOD model (Andre-Schmutz et al., Eur. J. Immunol., 29:245-255 (1999)). Little difference was detected in the pancreatic T2 values of control animals and those imaged on Day 1/2 after treatment with CPA (FIG. 8a). In both cases, the T2 response was as expected in the absence of pancreatic inflammation. It dropped below the baseline value immediately after MION injection due to the presence of circulating iron oxide within the bloodstream, but had recovered to baseline by 24 hours, reflecting an absence of nanoparticle leakage across the intact endothelial barrier.

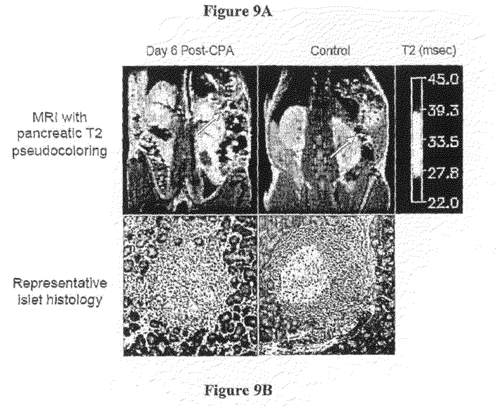

In contrast, striking alterations in the pancreatic T2 values were apparent on Days 3/4 and 5/6 after CPA treatment (plotted in FIG. 8a, visualized in FIG. 9). On these days, the values before and immediately after MION injection had increased markedly. This augmentation probably reflects the fact that baseline T2 is sensitive to alterations in ‘free water’, the increase in this instance probably signifying edema within the organ. The T2 value on these days had substantially dropped by 24 hours after MION injection, indicating that the probe had extravasated from the inflamed pancreatic microvasculature. The resulting accumulation of iron oxide within the pancreas, most likely within macrophages, altered its local magnetic characteristics, and thereby decreased its total T2 value below that of most other days studied, despite the effects of increased T2 from edema at these time-points. On Day 6, most of the islets had lost their characteristic structure due to massive lymphocytic infiltration, and this disintegration was accompanied by edema and separation of the surrounding acinar lobules (FIG. 9).

Mice were then scanned on subsequent days after CPA treatment, when β-cell destruction was complete and the local immune response resolving (FIG. 8a). On Days 14/15, the pancreatic T2 values at baseline and immediately following MION injection were indistinguishable from those of controls, suggesting that the edema had resolved. However, the T2 values at 24 hours after MION injection remained below baseline, indicating that pancreatic inflammation continued to enhance microvascular leak in these animals. On Days 35/36 after CPA-treatment, pancreatic T2 values had completely returned to the control level, signifying that at this late stage after islet destruction, microvascular integrity had returned to the normal, non-inflamed state. Importantly, the inflammatory changes were specific to the pancreas, as no alterations in T2 values were observed in other tissues, such as muscle (FIG. 8b).

Inflammation can be accompanied by a range of microvascular changes, including transient vasoconstriction, vasodilatation, increased blood flow and vascular leakage (Pober and Cotran, Transplantation, 50:537-544 (1990)). One of the powers of MNP-MRI is the ability to precisely quantify the relative contribution of two important microvascular parameters. First, this imaging strategy can measure alterations in VVF of the pancreas, caused by acute vasodilation or vasoconstriction, by comparing the baseline T2 value with the T2 obtained immediately after MION injection (Bremer et al., Radiology, 226:214-220 (2003)). Second, comparison of the baseline T2 value with the T2 measured 24 hours after MION injection yields information about vascular leakiness. The VVF of the pancreas remained unchanged during the evolution of CPA-induced diabetes in BDC2.5/NOD mice (FIG. 8c). In contrast, pancreatic microvascular leak followed a crescendo-decrescendo pattern, peaking at the time of maximal islet destruction (FIG. 8e). No significant alterations in either VVF or microvascular leak were observed in the muscle (FIGS. 8d and 8f).

Thus, this MNP-MRI approach allows one to image, non-invasively from live mammals, the major morphological features that accompany the development of autoimmune diabetes.

### Example 6

### Alterations in Pancreatic Inflammation Associated with Spontaneous Autoimmune Diabetes

Given the interest in eventually imaging the development of autoimmune diabetes in humans, the more complex, and therefore more clinically relevant, NOD mouse model was used next. The critical features of human type-1 diabetes, especially its genetic and immunological aspects, are very similar to those of the NOD disease, making this strain currently the most popular model for studying autoimmune diabetes. Since the T cell repertoire in standard NOD mice is much less skewed towards β-cell reactivity than in the engineered BDC2.5/NOD model, it was hypothesized that the inflammatory signal during the spontaneous development of autoimmune diabetes in NOD animals would be more subtle.

The questions that were addressed were: do mice recently diagnosed with spontaneous autoimmune diabetes have more pancreatic inflammation than do non-diabetic animals? And, is it possible to differentiate new-onset diabetic animals from those at risk of developing autoimmune diabetes based on non-invasive measures of pancreatic microvascular leakiness? Three groups of mice were compared in order to address these questions. Female NOD mice with new-onset diabetes (n=9), defined as hyperglycemia of less than 7 days duration, were compared with age-matched, non-diabetic female littermates (n=7); Eα16/NOD mice (n=13) served as a negative control. These animals have seemingly normal T and B lymphocyte compartments, but are completely protected from insulitis and the subsequent development of autoimmune diabetes through transgene-induced expression of the E complex on all cells normally displaying MHC class II molecules (Böhme et al., Science, 249:293-295 (1990)).

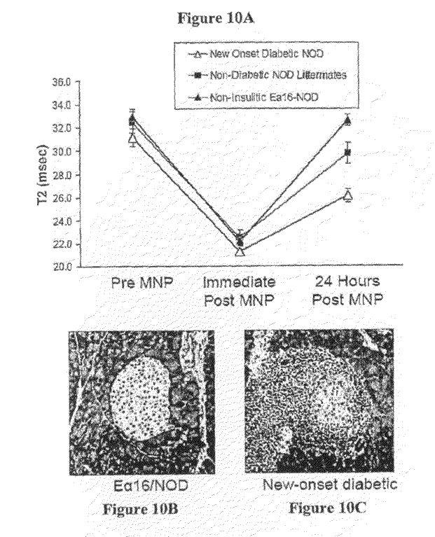

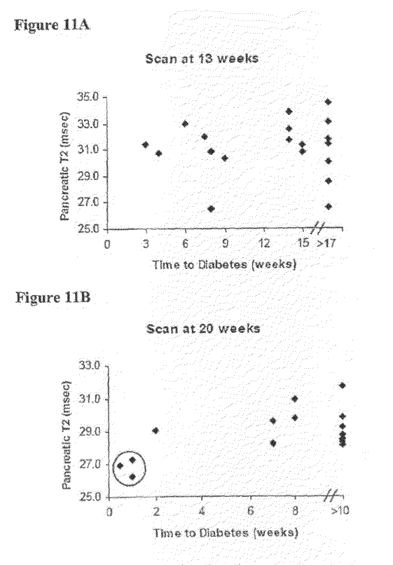

The pancreatic T2 values obtained at baseline and immediately after MION injection were indistinguishable in the three groups (FIG. 10a). These similarities in NOD mice contrast with the alterations seen in the BDC2.5/NOD animals, indicating that pronounced pancreatic edema is not a general feature of spontaneous autoimmune diabetes, but is rather a particularity of the exaggerated TCR Tg model. Interesting differences became apparent only 24 hours after MION administration, demonstrating that diabetes in NOD mice also is associated with alterations in microvascular leakiness, rather than in pancreatic VVF caused by vasodilation or vasoconstriction. T2 values in the non-insulitic Eα16/NOD mice (FIG. 10b) recovered to baseline 24 hours after MION injection, indicating that there was no significant vascular leak in the absence of insulitis. In contrast, the T2 in heavily infiltrated new-onset diabetic animals (FIG. 10c) remained well below the baseline value, and was significantly lower (and hence vascular leak was higher) than in both the non-insulitic Eα16/NOD group (P=0.00004) and non-diabetic NOD littermates (P=0.01). The latter mice, which have insulitis of variable severity, there being significant individual-to-individual variation in disease progression in the standard NOD model, had T2s that fell between those of non-insulitic Eα16/NOD animals (P=0.05) and the new-onset diabetic animals (P=0.01).

Thus, the evolution of pancreatic inflammation during the development of spontaneous diabetes can be monitored non-invasively in living subjects using a method described herein.

### Example 7

### Non-Invasive MRI to Predict the Eventual Development of Diabetes?

Extensive effort has focused on identifying markers that signal the imminent conversion of preclinical insulitis to clinical diabetes. To date, for both the NOD mouse model and human patients, the serum titers of autoantibodies directed against a defined set of islet-cell antigens have proven to be the most reliable indicators, as highlighted by recent results from the DPT-1 trial (N. Engl. J. Med., 346:1685-1691 (2002); Eisenbarth et al., Autoimmun. Rev., 1:139-145 (2002)). However, autoantibodies are at best an indirect measure of pancreas inflammation, and a non-invasive means of directly following diabetes progression in vivo would have a number of important applications.

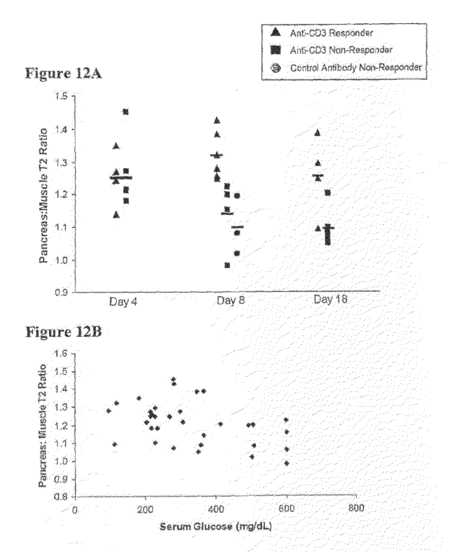

Therefore, a cohort of non-diabetic female NOD mice was scanned at 13 and 20 weeks of age, and the group was followed for the spontaneous development of diabetes up until 30 weeks. Unfortunately, it seems that this imaging approach is unlikely to be useful for prediction of diabetes risk, at least in the long-term time-frame tested here (FIG. 11). Data from the scan performed at 13 weeks showed no correlation between the pancreatic T2 value 24 hours after MION injection and the time at which diabetes was eventually diagnosed (FIG. 11a). However, some interesting insights were gained from the 20-week scans (FIG. 11b). The three mice with the lowest pancreatic T2 values (reflecting the highest microvascular leak) recorded at the 20-week scan all became diabetic within one week of the scan (circled in FIG. 11b). In contrast, mice that became diabetic at later times had T2 values essentially indistinguishable from the group that remained normoglycemic beyond 30 weeks.

Together, these data suggest that non-invasive monitoring of microvascular leakage provides important information concerning ‘acute’ inflammation of the pancreas, i.e., that associated with recent-onset or impending diabetes.

### Example 8

### Monitoring Alterations in Pancreatic Inflammation Following Anti-CD3 Monoclonal Antibody Treatment

A major potential application of this imaging technique is in monitoring acute changes in pancreatic inflammation in patients undergoing trial interventions to treat or prevent type-1 diabetes. The long half-life of islet autoantibodies renders them unresponsive to acute changes in autoimmune attack of the pancreas. In contrast, microvascular permeability is likely to change rapidly with successful immunointervention.

Short-term treatment of NOD mice with a monoclonal antibody (mAb) directed against the CD3ε subunit of the TCR reverses recent-onset diabetes and restores self-tolerance to β-cell antigens in a variable proportion of animals (Chatenoud et al., Proc. Natl. Acad. Sci. USA, 91:123-127 (1994); Chatenoud et al., J. Immunol., 158:2947-2954 (1997); Belghith et al., Nat. Med., 9:1202-1208 (2003)). Mice responding to this treatment generally return to normoglycemia within two to four weeks. The rate of diabetes remission varies with disease severity and is influenced by multiple factors, including the breeding facilities employed (Bach and Chatenoud, Annu. Rev. Immunol., 19:131-161 (2001)); our protocol generally results in a remission rate of 40-60%. Imaging this therapeutic regime in mice is particularly attractive because an analogous approach has been applied clinically using a non-activating humanized mAb against CD3, and was found to mitigate the deterioration in insulin production and to improve metabolic control in patients for up to 12 months following treatment(Herold et al., N. Engl. J. Med., 346:1692-1698 (2002)).

Anti-CD3 mAbs were purified from the supernatant of 145-2C11 hamster B cell hybridoma (American Type Culture Collection (ATCC), USA) grown under the recommended conditions. F(ab′)₂ fragments were generated (ImmunoPure F(ab′)₂ Preparation Kit, Pierce, USA) according to the manufacturer's protocol and purity was confirmed by SDS-PAGE gel electrophoresis. Anti-CD3 mAb therapy was commenced within 1 week of NOD mice developing spontaneous diabetes. Mice with new-onset diabetes received an intravenous injection of 50 μg 145-2C11 F(ab′)₂ fragments per day for five consecutive days (Chatenoud et al., J. Immunol., 158:2947-2954 (1997)). Mice were considered to have responded to therapy if blood glucose levels fell to the normal range and glycosuria resolved, and these benefits were maintained for at least 2 weeks. F(ab′)₂ fragments prepared from hamster monoclonal antibodies against DNP-derivatized keyhole limpet hemocyanin (UC8-1B9, ATCC, USA) were used for control studies.

To specifically monitor the phases previously shown to reflect the restoration of immunological self-tolerance, MR analysis of mice was performed on Days 4, 8 and 18 following initiation of anti-CD3 mAb therapy (FIG. 12a). On Days 4-8, the pancreas in mice responding favorably to treatment should be cleared of infiltrate; on day 18, the lymphocytes should have re-appeared, but should be accumulating in a peri-islet pattern. At these early time-points, it is often unclear clinically which animals have responded favorably to the immunointervention. Since some animals in this study remained profoundly hyperglycemic for longer than three weeks (i.e., the anti-CD3 non-responder group imaged at Day 18), the pancreas:muscle T2 ratio was calculated in mice scanned 24 hours after MION injection in order to control for any non-specific changes in MR parameters caused by these metabolic abnormalities. On Day 4 of the five-day treatment course, no difference between animals that eventually responded to anti-CD3 therapy by long-term restoration of normoglycemia and those that failed to respond could be detected (number of responders=4; number of non-responders=6). On Day 8 after initiation of anti-CD3 therapy, mice with a favorable response had significantly less microvascular leak in the pancreas than either the animals that did not respond to therapy (P=0.01) or those treated with a control mAb (P=0.005. Number of responders=6; number of non-responders=4; number of control mAb treated=3). These differences were still detectable at Day 18, although the magnitude of the difference was less (P=0.02. Number of responders=4; number of non-responders=6).

To address the possibility that pancreatic inflammation as measured by MNP-MRI merely correlated with glycemic control, the Pancreas:Muscle T2 ratio was plotted against serum glucose at the time of scanning for all individuals in this experimental series (n=33). There was marked variation in Pancreas:Muscle T2 ratio at all levels of serum glucose (FIG. 12b), and hence, no relationship between degree of pancreatic inflammation and level of hyperglycemia.

Thus, MR-based imaging can be used to non-invasively monitor changes in pancreatic inflammation following treatment with a mAb targeting CD3. Moreover, MNP-MRI was able to identify subjects with a favorable response to therapy as early as three days after completing the course of anti-CD3 treatment (i.e., Day 8).

### Example 9

### In Vivo Imaging of Microvascular Leakage in Human Subjects

This example describes the use of methods described herein to image insulitis in living human subjects. All subjects were treated in accordance with the institutional review board's guidelines and gave their informed consent to participate in the study.

At the time of their visit for the scan, when the IV was being placed, all subjects had serum drawn for determination of GAD, IA-2 and IAA. Subjects without diabetes had an oral glucose tolerance test (OGTT) with glucose and insulin values determined within 6 weeks of the time of the scan. HLA-DQ typing was performed on subjects of unknown genotype. Autoantibody testing and OGTT with insulin and C-peptide values were performed. Recent data from the DPT-1 study have shown that the two hour C-peptide identifies relatives at increased risk for type-1 diabetes, similar to the degree of risk associated with lower one and three minute insulin values on an IVGTT (intravenous glucose tolerance test). OGTT glucose and C-peptide values were used as metabolic parameters in these studies. Subjects who were undergoing scanning for other studies were used as a comparison group.

MR imaging was performed on a 1.5 T clinical imaging system (System 5X, General Electric Medical Systems, Milwaukee, Wis.) using a pelvic phased array coil or another appropriate imaging coil. The MR imaging included conventional T1 and T2 weighted spin echo and 3 D gradient echo sequences.

Each subject underwent three scans. The scanning parameters were optimized by the MGH imaging group. For each subject, three scans were performed. At the initial visit a pre-scan was done, followed by a Combidex® infusion at dose of 2.6 mg Fe/kg, and then a post- scan was done immediately after. This post-scan was to obtain vascular volume data. A third scan was performed the following day at the second visit to obtain permeability data.

Supplied Combidex® in vials was reconstituted with 10 mL of normal saline. The reconstituted contrast was administered to each subject at a dose of 2.6 mg Fe/kg. Following reconstitution, an appropriate weight-specific dose of the contrast was drawn and further diluted with 100 mL of normal saline. Following dilution the agent was injected in a piggyback fashion through a filter at a rate of 4 ml per minute until the entire volume was infused.

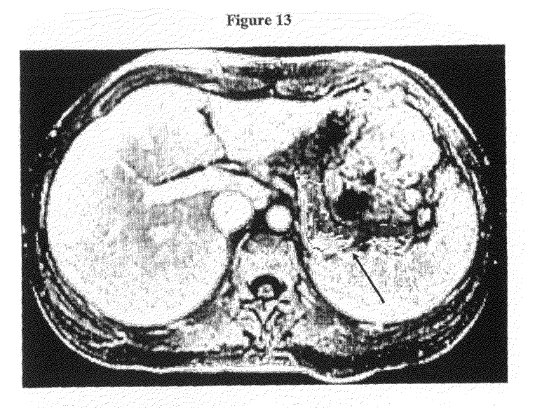

FIG. 13 is an image at the level of the upper abdomen in an individual who has had a hyperglycemic episode. An area of pancreatic inflammation that was highlighted by accumulation of the MIONs is shown in pseudocolor. These results indicate that the methods described herein are applicable to imaging pancreatic inflammation in human subjects.

### REFERENCE LIST

Eisenbarth et al., Autoimmun. Rev., 1:139-145 (2002)
Abiru et al., Diabetes Metab. Res. Rev., 18:357-366 (2002)
Naik and Palmer, Rev. Endocr. Metab. Disord., 4:233-241 (2003)
Pober and Cotran, Transplantation, 50:537-544 (1990)
Sandler and Jansson, Virchows Arch., 407:359-367 (1985)
Beppu et al., Acta Endocrinol., 114:90-95 (1987)
Martin et al., Diabetologia, 32:359-367 (1989)
Papaccio et al., Histochemistry, 95:19-21 (1990)
Papaccio and Chieffi-Baccari, Histochemistry, 97:371-374 (1992)
Papaccio, Histol. Histopath., 8:751-759 (1993)
Papaccio et al., Autoimmunity, 27:65-77 (1998)
Jansson and Sandler, Virchows Arch. A. Pathol. Anat. Histopathol., 410:17-21 (1986)
Carlsson et al., Endocrinology, 139:3534-3541 (1998)
Papaccio et al., J. Cell. Biochem., 86:651-664 (2002)
Bogdanov et al., Adv. Drug Deliv. Reviews, 37:279-293 (1999)
McDonald et al., Nat. Med., 9:713-725 (2003)
Weissleder et al., Nat. Biotechnol., 19:316-317 (2001)
Hogemann et al., Bioconjug. Chem., 11:941-946 (2000)
Harisinghani et al., N. Engl. J. Med., 348:2491-2499 (2003)
Katz et al., Cell, 74:1089-1100 (1993)
Haskins et al., Diabetes, 37:1444-1448 (1988)
Haskins et al., Proc. Natl. Acad. Sci. USA, 86:8000-8004 (1989)
Gonzalez et al., Immunity, 7:873-883 (1997)
Weissleder et al., Radiology, 175:489-493 (1990)
Weissleder et al., Radiology, 191:225-230 (1994)
Henderson et al., Q. J. Exp. Physiol., 70:347-356 (1985)
Moore et al., Radiology, 214:568-574 (2000)
Luhder et al., J. Exp. Med., 187:427-432 (1998)
Guo et al., J. Immunol., 166:1073-1086 (2001)
Bellhorn et al., Invest. Ophthalmol. Vis. Sci., 21:282-290 (1981)
Böhme et al., Science, 249:293-295 (1990)
Gonzalez et al., Nat. Immunol., 2:1117-1125 (2001)
Hoglund et al., J. Exp. Med., 189:331-339 (1999)
Jansen et a., Diabetes, 43:667-675 (1994)
Shinomiya et al., Pancreas, 20:290-296 (2000)
Murata et al., Eur. J. Immunol., 33:1001-1011 (2003)
Shen et al., Magn. Reson. Med., 29:599-604 (1993)
Josephson et al., Bioconjug. Chem., 10:186-191 (1999)
Larson et al., Science, 300:1434-1436 (2003)
Bach, Endocr. Rev., 15:516-542 (1994)
Atkinson and Eisenbarth, Lancet 358:221-229 (2001)
Tisch and McDevitt, Cell, 85:291-297 (1996)
Imagawa et al., Diabetes, 50:1269-1273 (2001)
N. Engl. J. Med., 346:1685-1691 (2002)
De Paepe et al., Diabetologia, 35:1118-1124 (1992)
Majno et al., Am. J. Pathol., 128:210-215 (1987)
Kitagawa et al., Immunopharmacology, 25:229-238 (1993)
McDonald and Choyke, Nat. Med., 9:713-725 (2003)
Bremer et al., Radiology, 226:214-220 (2003)
Weissleder et al., Nat. Med., 6:351-355 (2000)
Denis et al., Proc. Natl. Acad. Sci. USA, 101:12634-12639 (2004)
Andre-Schmutz et al., Eur. J. Immunol., 29:245-255 (1999)
Chatenoud et al., Proc. Natl. Acad. Sci. USA, 91:123-127 (1994)
Chatenoud et al., J. Immunol., 158:2947-2954 (1997)
Belghith et al., Nat. Med., 9:1202-1208 (2003)
Bach and Chatenoud, Annu. Rev. Immunol., 19:131-161 (2001)
Herold et al., N. Engl. J. Med., 346:1692-1698 (2002)
Signore et al., J. Endocrinol. Invest, 22:151-158 (1999)
Moore et al., Diabetes, 50:2231-2236 (2001)
Moore et al., Diabetes, 53:1459-1466 (2004)
Tree and Peakman, Endocrinol. Metab. Clin. North Am., 33:113-133, ix-x (2004)
Panagiotopoulos et al., Curr. Diab. Rep., 4:87-94 (2004)
Lieberman and DiLorenzo, Tissue Antigens, 62:359-377 (2003)
Jaeger et al., Horm. Metab. Res., 29:510-515 (1997)
Bougneres et al., N. Engl. J. Med., 318:663-670 (1988)
Palmer et al., Diabetes, 53:250-264 (2004)
Chatenoud, Nat. Rev. Immunol., 3:123-132 (2003)

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of imaging inflammation in a pancreatic tissue of a living mammal in vivo, the method comprising:
administering a detectable amount of a composition comprising Magnetic Nanoparticle Probes (MNPs) or derivatives thereof to a living mammal; and
detecting MNPs in the pancreatic tissue of the mammal,
thereby imaging inflammation in the pancreatic tissue.

2. The method of claim 1, wherein the presence of MNPs in the pancreatic tissue of the mammal is an indication of the presence of inflammation in the tissue.

3. The method of claim 1, wherein the MNPs have no targeting moiety.

4. The method of claim 1, wherein the MNPs are detected by NMR imaging.

5. The method of claim 1, wherein the living mammal is at risk of developing type-1 diabetes.

6. The method of claim 1, wherein the living mammal is selected on the basis of being at risk of developing type-1 diabetes.

7. The method of claim 1, wherein the amount of MNPs detected in the pancreatic tissue of the mammal indicates that the subject is developing or will develop type-1 diabetes.

8. The method of claim 1, wherein the living mammal is selected on the basis of being at risk of developing insulitis.

9. The method of claim 1, wherein the amount of MNPs detected in the pancreatic tissue of said mammal indicates that the subject is developing or will develop insulitis.

10. A method of evaluating the efficacy of a candidate treatment for pancreatic inflammation in a subject, the method comprising:
selecting a subject;
administering a candidate therapeutic intervention to the subject; and
obtaining an in vivo image of inflammation in a pancreatic tissue of the subject, wherein the presence, absence, or level of inflammation in the pancreatic tissue is indicative of the efficacy of the candidate treatment.

11. The method of claim 10, wherein the absence or level of inflammation indicates that the candidate treatment is an effective treatment for pancreatic inflammation.

12. The method of claim 10, further comprising obtaining an in vivo image of inflammation in a pancreatic tissue in the subject before administration of the candidate treatment.

13. The method of claim 10, wherein the treatment for pancreatic inflammation is a candidate treatment for type-1 diabetes.

14. The method of claim 10, wherein the treatment for type-1 diabetes prevents or delays the onset or progression of type-1 diabetes.

15. The method of claim 10, wherein the subject is selected on the basis of being at risk of developing type-1 diabetes.

16. The method of claim 10, wherein the in vivo image of a pancreatic tissue of the subject is obtained a method comprising:
administering a detectable amount of a composition comprising Magnetic Nanoparticle Probes (MNPs) or derivatives thereof to a living mammal; and
detecting MNPs in the pancreatic tissue of the mammal.

17. A method of evaluating pancreatic inflammation in a subject, the method comprising:
administering a detectable amount of a composition comprising Magnetic Nanoparticle Probes (MNPs) or derivatives thereof to the mammal;
obtaining a magnetic resonance image (MRI) of MNPs in a pancreatic tissue of the mammal; and
deriving a value relevant to inflammation from the image of MNPs;
thereby evaluating pancreatic inflammation in the subject.

18. The method of claim 17, wherein the MRI is obtained immediately after administration of the MNPs.

19. The method of claim 17, further comprising obtaining a second MRI.

20. The method of claim 19, further comprising obtaining a second MRI at least about 24 hours after administration of the MNPs.

21. The method of claim 17, wherein the value relevant to inflammation is selected from the group consisting of vascular volume fraction (VVF), vascular leak, T1, T2, and T2*.

22. The method of claim 17, further comprising comparing the relevant value with a reference value.

23. The method of claim 22, wherein the relevant value as compared with the reference value is indicative of a rate of progression of pancreatic inflammation in the subject.

24. The method of claim 22, wherein the reference value is derived from an image of MNPs in the pancreatic tissue of the mammal obtained previously.

25. The method of claim 1, wherein the MNPs are monocrystalline superparamagnetic iron oxide particles with a dextran coating.

26. The method of claim 1, wherein the MNPs are Monocrystalline Iron Oxide Nanoparticles (MIONs), or derivatives thereof.

27. The method of claim 10, wherein the MNPs are monocrystalline superparamagnetic iron oxide particles with a dextran coating.

28. The method of claim 10, wherein the MNPs are Monocrystalline Iron Oxide Nanoparticles (MIONs), or derivatives thereof.

29. The method of claim 17, wherein the MNPs are monocrystalline superparamagnetic iron oxide particles with a dextran coating.

30. The method of claim 17, wherein the MNPs are Monocrystalline Iron Oxide Nanoparticles (MIONs), or derivatives thereof.
